(19) United States
(12) Patent Application Publication
(10) Pub. No.: US 2003/0220288 A1
(43) Pub. Date: Nov. 27, 2003

(54) Nor-1 and nur77 nuclear receptors as targets for anti-leukemia therapy

(75) Inventors: Shannon E. Mullican, Houston, TX (US); Orla M. Conneely, Houston, TX (US); Jeffrey Milbrandt, St. Louis, MO (US)

Correspondence Address: FULBRIGHT & JAWORSKI, LLP, 1301 MCKINNEY, SUITE 5100, HOUSTON, TX, 77010-3095, US

(73) Assignee: Baylor College of Medicine

(21) Appl. No.: 10/414,080

(22) Filed: Apr. 15, 2003

Related U.S. Application Data
(60) Provisional application No. 60/373,238, filed on Apr. 17, 2002.

(51) Int. Cl. A61K 48/00; C12N 15/861

(52) U.S. Cl. 514/44; 424/93.2; 435/456

(57) ABSTRACT

The present invention is directed to the application of nuclear receptor transcription factors as molecular targets for therapeutic intervention in the treatment of myeloid leukemia. More specifically, nor-1 and nur77 nuclear receptors are targets for myeloid leukemia therapy.

This patent application claims priority to U.S. Provisional Application, Serial No. 60/373,238, filed Apr. 17, 2002, which is incorporated by reference herein in its entirety.

### STATEMENT REGARDING FEDERALLY SPONSORED RESEARCH OR DEVELOPMENT

The present invention was developed using funds from NIH Grant No. DK57743. The United States Government may have certain rights in the invention.

### FIELD OF THE INVENTION

The field of the present invention generally includes cell biology, molecular biology, and cancer therapy, such as for leukemia. More particularly, the present invention regards nor-1 and nur77 nuclear receptors as targets for anti-leukemic drug intervention.

### BACKGROUND OF THE INVENTION

Leukemia is a type of cancer that is defined as an excessive production of cells of bone marrow origin (hematopoietic cells). Leukemia is further classified based on the specific cell lineage that is affected (lymphoid and myeloid). The detrimental effects of the uncontrolled production of these cells include altered development of other hematopoietic cell lineages and infiltration into peripheral tissues, such as the lung, that can affect the normal function of that organ, and may ultimately lead to death.

Nor-1 (NR4A3, TEC, MINOR, CHN) and nur77 (NR4A1, TR3, NGFI-B, NAK1, HMR) are members of the Nuclear Receptor Superfamily. Nuclear receptors are transcription factors that are activated by binding small molecule ligands. Ligand binding induces conformational changes in nuclear receptors that allow them to recruit coregulator proteins to the transcription apparatus to induce transcription of specific genes. Transcriptional regulation activity of nor-1 and nur77 can also be regulated by binding of co-factors, or posttranslational modifications such as phosphorylation induced by signaling cascades as a result of cell exposure to stimuli such as but not limited to growth factors, neurotransmitters, cyclic AMP, cytokines, or mechanical stimulation. One well-studied area where nor-1 and nur77 have been thought to play a role is in the development of t-lymphocytes. T-lymphocytes originate in the bone marrow and then migrate to the thymus where they undergo the majority of their maturation. It is known that nor-1 and nur77 are necessary for a process called negative selection within the thymus. Negative selection causes t-lymphocytes reactive to self-proteins to die before they enter the periphery, and it occurs late in t-lymphocyte development. This process is important in preventing autoimmunity.

Wu et al. (2002) describes interaction and inhibition of Nur77 by the promyelocytic leukemia protein (PML) in a dose-dependent manner. Specifically, the coiled-coil domain of PML interacts with the DNA-binding domain of Nur77 (amino acids 267-332). The data is described in the context of supporting a role for PML/Nur77 interaction in regulating cell growth and apoptosis.

Bandoh et al. (1997) demonstrate that mechanical agitation transiently induced nor-1, ngfi-b (nur77), and nurr1 mRNAs in several leukemic cell lines in a dose-dependent manner, particularly in the HL-60 promyelocytic leukemia cell line.

Thus, during hematopoiesis, prior to the negative selection stage of t-lymphocytes, no role of lymphocyte development has been assigned to either nor-1 or nur77. The present invention addresses such a finding and provides methods and compositions useful for leukemia prevention and therapy.

### BRIEF SUMMARY OF THE INVENTION

The present invention is directed to methods and compositions related to nuclear receptors nor-1 and/or nur77 for therapy and prevention of leukemia, particularly myeloid leukemia with differentiation.

In an embodiment of the present invention, there is a method of inhibiting proliferation of a hematopoietic cell, comprising the step of modulating the level of nor-1 and/or nur77 nuclear receptor. In some embodiments, the hematopoietic cell may be a hematopoietic stem cell or a hematopoietic myeloid cell. The modulating step may be defined as increasing the level of a nor-1 and/or nur77 nuclear receptor polypeptide, and the increasing step may be defined as increasing the level of a nor-1 and/or nur77 nuclear receptor polynucleotide.

In some embodiments, the nor-1 and/or nur77 nuclear receptor polynucleotide is increased through administration of a vector comprising the polynucleotide, and the vector may be a viral vector or a non-viral vector. Viral vector includes an adenoviral vector, a retroviral vector, or an adeno-associated vector. In a specific embodiment, the viral vector is an adenoviral vector. In another specific embodiment, the non-viral vector is a plasmid. In another specific embodiment, the nor-1 and/or nur77 nuclear receptor polynucleotide is increased through upregulation of expression. In a further specific embodiment, the upregulation of expression is of the nor-1 and/or nur77 nuclear receptor, and the upregulation of expression of the nor-1 and/or nur77 nuclear receptor may be through administration of growth factors, cytokines, cyclic AMP, or a mixture thereof. In some embodiments, the cell is in a mammal afflicted with leukemia.

In another embodiment of the present invention, there is a method of inhibiting proliferation of a hematopoietic cell, comprising the step of modulating the activity of a nor-1 and/or nur77 nuclear receptor. The hematopoietic cell may be a hematopoietic stem cell or a hematopoietic myeloid cell. In some embodiments, the modulating step is defined as increasing transcriptional activity of a nor-1 and/or nur77 nuclear receptor polypeptide. In other embodiment of the present invention, the modulating step is further defined as administering an agonist to the nor-1 and/or nur77 nuclear receptor polypeptide.

In an additional embodiment of the present invention, there is a method of treating leukemia in an individual, comprising the step of modulating a nor-1 and/or nur77 nuclear receptor in the individual. In a specific embodiment, the modulating step occurs in a hematopoietic cell of the individual. The hematopoietic cell may be a hematopoietic stem cell or a hematopoietic myeloid cell.

In an additional embodiment of the present invention, the modulating step is further defined as increasing the activity of a nor-1 and/or nur77 nuclear receptor polypeptide, is further defined as increasing the level of a nor-1 and/or nur77 nuclear receptor polypeptide, or is further defined as increasing the level of a nor-1 and/or nur77 nuclear receptor polynucleotide.

In a further specific embodiment of the present invention, the increasing activity step is further defined as introducing an agonist to said nor-1 and/or nur77 nuclear receptor polypeptide. In some embodiments, the introducing step is further defined as administering said agonist in a pharmaceutically acceptable composition to said individual, such as a ligand of said nor-1 and/or nur77 nuclear receptor, although the agonist may not be a ligand of said nor-1 and/or nur77 nuclear receptor.

In some embodiments, the increasing the level of a nor-1 and/or nur77 nuclear receptor polynucleotide step is defined as increasing expression of a respective nor-1 and/or nur77 nuclear receptor in a cell of the individual. The cell may be a hematopoietic bone marrow stem cell or a hematopoietic myeloid cell. In an additional specific embodiment, the increasing the level of a nor-1 and/or nur77 nuclear receptor polynucleotide step is defined as increasing the half-life of a respective nor-1 and/or nur77 nuclear receptor mRNA in a cell of the individual. In a further specific embodiment, the method further comprises the step of administering said cell to an individual.

In an additional embodiment of the present invention, there is a method of increasing the level of a nor-1 and/or nur77 nuclear receptor in a hematopoietic cell, comprising the step of administering a compound to the cell to increase the expression of said nor-1 and/or nur77 nuclear receptor. The compound may be a growth factor, cytokine, cyclic AMP, or a mixture thereof. The method may be further defined as administering said compound in a pharmaceutically acceptable composition to said individual.

In another embodiment of the present invention, there is a method of identifying an upregulator of expression of a nor-1 and/or nur77 nuclear receptor, comprising the steps of introducing to a cell a test agent, wherein the cell comprises a marker sequence and wherein the expression of the marker sequence is regulated by a nor-1 and/or nur77 nuclear receptor regulatory sequence; and measuring for an increase in the expression level of the marker sequence, wherein when the increase occurs following introduction of said test agent to the cell, the test agent is the upregulator. In a specific embodiment of the present invention, the method further comprises administering the upregulator in a pharmaceutically acceptable composition to an individual. In another specific embodiment, the individual is susceptible to leukemia or is diagnosed with leukemia.

In an additional embodiment of the present invention, there is a method of identifying a compound for the treatment of leukemia, comprising the steps of obtaining a compound suspected of having activity of a nor-1 and/or nur77 nuclear receptor agonist; and determining whether said compound has said activity. In a specific embodiment, the agonist is a ligand of a nor-1 and/or nur77 nuclear receptor. The method may further comprise dispersing the compound in a pharmaceutical carrier; and administering a therapeutically effective amount of the compound in the carrier to an individual having leukemia.

In an additional embodiment of the present invention, there is a compound obtained by a method described herein.

In another embodiment of the present invention, there is a pharmacologically acceptable composition comprising the compound obtained by a method described herein and a pharmaceutical carrier.

In an additional embodiment of the present invention, there is a method of screening for a compound for the treatment of leukemia, comprising the steps of providing a first vector comprising a nor-1 or nur77 nucleic acid sequence encoding a respective nor-1 or nur77 gene product, wherein the expression of said nor-1 or nur77 nucleic acid sequence is under the control of a first regulatory sequence; providing a second vector comprising a reporter nucleic acid sequence encoding a reporter gene product, wherein the expression of said reporter nucleic acid sequence is under the control of a second regulatory sequence, wherein the second regulatory sequence is responsive to nor-1 or nur77; providing a test agent; providing a leukemia cell line, wherein cells in said cell line comprise conditions suitable for expression of said nor-1 or nur77 gene product and said reporter gene product; and assaying transcriptional regulation activity of said nor-1 or nur77 gene product by measuring expression or activity of the reporter gene product in the presence of said test agent, wherein when the expression or activity of the reporter gene product changes in the presence of the test agent, the test agent is the compound for the treatment of leukemia. The leukemic cell line may be K562, U937, AML-193, HL-60, LSTRA, or CEM. In a specific embodiment, the first vector, second vector, test agent, or a combination thereof are introduced into the cell line. In another specific embodiment, the reporter nucleic acid is β-galactosidase, green fluorescent protein, blue fluorescent protein, or chloramphenicol acetyltransferase. The expression or activity of the reporter gene product increases in the presence of the test agent, in some embodiments.

In an additional embodiment of the present invention, there is a mouse model for leukemia, comprising a mouse having defective nor-1 and/or nur77 nucleic acid sequences. In a specific embodiment, the mouse is further defined as having a knockout mutation in the genes encoding nor-1 and nur77, respectively. In another specific embodiment, the mouse is further defined as having the nor-1KO/nur77± genotype, the nor-1±/nur77 KO, or the nor-1KO/nur77KO, wherein KO is defined as a knockout. In a further specific embodiment, the mouse comprises at least one symptom of leukemia.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 depicts a growth curve representing several litters that were weighed daily for a period of 14 days.

FIG. 2 illustrates lymphadenopathy and splenomegaly observed in the nor-1KO (knockout)/nur77KO mice. Tissues on the right in both panels are from normal littermates.

FIG. 3 is an illustration of liver discoloration observed in the nor-1KO/nur77KO mice (right side is normal littermate).

FIG. 4 illustrates altered histology of the spleen and thymus in the nor-1KO/nur77KO mice. Left panels show tissue from normal littermates.

FIG. 5 shows abnormal presence of medullary epithelial cells throughout the nor-1KO/nur77KO thymus (right panel shows normal thymus).

FIG. 6 demonstrates the total number of thymocytes are reduced in the nor-1KO/nur77KO mice.

FIG. 7 illustrates reduction in total thymocyte number is not limited to any specific CD4/CD8 developmental stage.

FIG. 8 shows perivascular cellular infiltrates in the liver, lung, and pancreas of the nor-1KO/nur77KO mice. The left hand panels show tissues from normal littermates.

FIG. 9 demonstrates CD11b/Gr-1 expressing cells are increased in the nor-1KO/nur77KO lymphoid tissues and blood. Dotplots from normal littermates are shown on the left.

FIG. 10 shows positive myeloperoxidase staining in the nor-1KO/nur77KO perivascular cellular infiltrates and lymphoid tissues.

FIG. 11 shows cells within the perivascular infiltration and lymphoid tissue in the nor-1KO/nur77KO mice are CD11b positive.

FIG. 12 demonstrates abnormal hematopoiesis in the bone marrow of the nor-1KO/nur77KO mice (the left panels are bone marrow results from normal littermates.)

FIG. 13 shows hypoallelic nor-1KO/nur77± mice display abnormal lymphoid tissue architecture.

FIG. 14 demonstrates perivascular cell infiltrates in the hypoallelic nor-1KO/nur77± mouse.

### DETAILED DESCRIPTION OF THE INVENTION

Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The term “agonist” as used herein is defined as a factor that promotes, facilitates or enhances the activity or function of another biological entity. In a specific embodiment, the agonist is an agonist of transcription regulatory activity of the nor-1 or nur77 polypeptide. The agonist may be a small molecule, an amino acid sequence, a nucleic acid sequence, a lipid, a sugar, a carbohydrate, polypeptide, or a combination thereof.

The term “anti-leukemic activity” as used herein is defined as having activity that improves, at least in part, one or more symptoms of myeloid leukemia. Symptoms are well known in the art, however, some examples include excessive production of cells of bone marrow origin (hematopoietic cells) of the myeloid lineage, altered development of other hematopoietic cell lineages and/or infiltration into peripheral tissues, anemia, and splenomegaly.

The term “gene product” as used herein is defined as a mRNA, a polypeptide or both an mRNA or polypeptide encoded by a nucleic acid sequence.

The term “ligand” as used herein is defined as a molecule that binds to another molecule, preferably a receptor, and more preferably a nuclear-localized receptor. In a specific embodiment, a ligand that binds to nor-1 and/or nur77 is preferred. One skilled in the art recognizes that a ligand includes the whole ligand, or any part or any mutant thereof that remains capable of binding to nor-1 and/or nur77.

The term “modulating” as used herein is defined as altering the level, activity, or both of nor-1 and/or nur77 nuclear receptor polypeptide.

The term “non-ligand agonist” as used herein is defined as an agonist that does not directly bind the receptor but enhances its biological activity by either increasing the cellular level of nor-1 and/or nur77 or activation of nor-1 and/or nur77 protein by covalent modification such as phosphorylation.

The term “therapeutically effective” as used herein is defined as the amount of a compound required to improve some symptom associated with a disease. For example, in the treatment of leukemia, a compound that decreases, prevents, delays or arrests any symptom of the disease would be therapeutically effective. A therapeutically effective amount of a compound is not required to cure a disease. A compound is to be administered in a therapeutically effective amount if the amount administered is physiologically significant. A compound is physiologically significant if its presence results in technical change in the physiology of a recipient organism.

The term “upregulator” as used herein is defined as a compound that indirectly or directly causes an increase in expression of nor-1 and/or nur77 nuclear receptors.

The Present Invention

Nor-1 and nur77 nuclear receptors are redundant in the process of negative selection in the thymus. Therefore, deletion of either of these genes in mice does not result in altered t-lymphocyte development. The inventors predicted based on previous studies that deletion of both nor-1 and nur77 would result in a defect in the later stages of t-lymphocyte development, specifically negative selection. Unexpectedly, as shown herein, upon deletion of both of these nuclear receptors, mice do not survive past 4 weeks of age. Also unexpectedly, in addition to a defect during the stage of negative selection, lymphocyte development is also altered during the earlier stages. This early defect in lymphocyte development is secondary to a severe overproduction of myeloid cells in the bone marrow leading to myeloid leukemia with differentiation in the mice lacking both nor-1 and nur77 (nor-1KO/nur77KO). No previous reports have implicated either nor-1 or nur77 in bone marrow hematopoiesis in the myeloid lineage or in the prevention of development of leukemia. The results that support the diagnosis of myeloid leukemia in these mice are summarized herein and reflect the novel aspect of the present invention regarding leukemia prevention and treatment.

It is an object of the present invention to relate methods of treatment, methods of prevention, agonists and other compositions to nor-1 and/or nur77 for leukemia. In one aspect of the present invention, both nor-1 and nur77 are within the scope of the present invention, particularly given the striking structurally and genetically related redundancy of these two family members. A skilled artisan recognizes that these genes may also be referred to as being in the Nur nuclear receptor superfamily or the NGFI-B subfamily of a nuclear receptor superfamily. Characteristics of nor-1 and/or nur77 may include a central DNA binding domain comprising two highly conserved zinc finger motifs (Berg, 1989; Klug and Schwabe, 1995), a ligand-binding domain comprising 8-9 heptad repeats of hydrophobic amino acids in the carboxyl terminus, and/or a variable amino-terminal region.

One skilled in the art recognizes that within the scope of the invention a NOR-1 sequence is utilized. Examples of nucleic acid NOR-1 sequences comprise SEQ ID NO: 1 (1651190), SEQ ID NO: 2 (D38530). SEQ ID NO: 3 (AF050223), SEQ ID NO: 4 (BG235965), SEQ ID NO: 5 (BE65671 1), SEQ ID NO: 6 (AJ011768), SEQ ID NO: 7 (E14965; a useful exemplary Nor1 promoter region), SEQ ID NO: 8 (AJ011767), SEQ ID NO: 9 (D85244, another exemplary Nor1 promoter region), SEQ ID NO: 10 (D85243), SEQ ID NO: 11 (D85242), and SEQ ID NO: 12 (D85241).

Examples of amino acid NOR-1 sequences comprise SEQ ID NO: 13 (7441771), SEQ ID NO: 14 (Q92570), SEQ ID NO: 15 (JC2493), SEQ ID NO: 16 (CAA09764), SEQ ID NO: 17 (CAA09763), SEQ ID NO: 18 (BAA31221), and SEQ ID NO: 19 (BAA28608).

One skilled in the art recognizes that within the scope of the invention a NUR77 sequence is utilized. Examples of nucleic acid NUR77 sequences comprise SEQ ID NO: 20 (1339917), SEQ ID NO: 21 (12662548), SEQ ID NO: 22 (BF937382), SEQ ID NO: 23 (BE198460), SEQ ID NO: 24 (BE047656), SEQ ID NO: 25 (BE047651), SEQ ID NO: 26 (AW988827), SEQ ID NO: 27 (AA461422), SEQ ID NO: 28 (D49728), and SEQ ID NO: 29 (S77154).

Examples of amino acid NUR77 sequences comprise SEQ ID NO: 30 (127819), SEQ ID NO: 31 (128911), SEQ ID NO: 32 (P22829), SEQ ID NO: 33 (AAB33999), SEQ ID NO: 34 (AAA42058), and SEQ ID NO: 35 (A37251). A skilled artisan would know how to retrieve sequences from the National Center for Biotechnology Information's Genbank database or commercially available databases such as the genetic database by Celera Genomics, Inc. (Rockville, Md.).

In the present invention, the methods are used for treating and/or preventing leukemia, particularly myeloid leukemia. Examples of use in the treatment would be for the improvement of the disease after its onset or in helping alleviate at least one symptom. The disease is considered to be improved if at least one symptom is alleviated, wherein alleviation may be partial or complete. Symptoms to be alleviated include but are not limited to increased white blood cells in the peripheral blood, altered hematopoietic lineages in the bone marrow, anemia, splenomegaly, hematopoietic infiltration into peripheral non-hematopoietic tissues, etc. An example of use for the prevention of the disease would be the use prior to the onset of leukemia, and thus, prevent or delay its onset.

One specific embodiment of the present invention is a method of preventing or treating leukemia comprising the step of modulating nor-1 and/or nur77, such as its function or level. In a specific embodiment, nor-1 and/or nur77 receptor polynucleotide is increased, such as by upregulation of its expression or by increase of the mRNA transcription. In another specific embodiment, nor-1 and/or nur77 nuclear receptor polypeptide level is increased or the activity of nor-1 and/or nur77 nuclear receptor polypeptide is enhanced or facilitated, or both polypeptide level is increased and activity is enhanced. One skilled in the art recognizes that there are a variety of ways to increase nor-1 and/or nur77 nuclear receptor levels, such as administering to a cell one or more nor-1 and/or nur77 nuclear receptor polypeptides or to upregulate expression of a nor-1 and/or nur77 nuclear receptor polynucleotide. Furthermore, a skilled artisan recognizes how to enhance the activity of nor-1 and/or nur77 nuclear receptor polypeptides, such as by introducing an agonist to the polypeptide, either directly or indirectly. In other embodiments, a nor-1 or nur77 nuclear receptor polynucleotide is delivered to a cell to increase level of the nor-1 or nur77 nuclear receptor polynucleotide and/or polypeptide, and in specific embodiments the cell is comprised in an individual.

In particular embodiments, the expression of the nor-1 and/or nur77 nuclear receptor is upregulated, wherein the upregulation results indirectly or directly with inhibiting proliferation of a hematopoietic cell, such as a hematopoietic stem cell, a hematopoietic myeloid cell, or both. In specific embodiments, the upregulation in expression is a result of administration of a factors such as but not limited to growth factors, cytokines, cyclic AMP, or a mixture thereof. Examples of growth factors include but are not limited to epidermal growth factor, hematopoietic stem cell growth factor (SCGF) (such as is described in U.S. Pat. No. 6,541,217, incorporated by reference herein in its entirety), granulocyte macrophage-colony stimulating factor (GM-CSF), granulocyte-colony stimulating factor (G-CSF), macrophage-colony stimulating factor (M-CSF), tumor necrosis factors (TNF.alpha. and TNF.beta.), transforming growth factors (TGF.alpha. and TGF.beta.), stem cell factor (SCF), platelet-derived growth factors (PDGF), nerve growth factor (NGF), fibroblast growth factors (FGF), insulin-like growth factors (IGF-I and IGF-II), growth hormone, interleukin-1, interleukin-2, keratinocyte growth factor, ciliary neurotrophic growth factor, Schwann cell-derived growth factor, and vaccinia virus growth factor. Examples of cytokines include but are not limited to IL-1α, IL-1β, IL-2, IL-3, IL-4, IL-5, IL-6, IL-7, IL-8, IL-9, IL-10, IL-11, IL-12, IL-13, TGF-β, GM-CSF, M-CSF, G-CSF, TNF-α, TNF-β, LAF, TCGF, BCGF, TRF, BAF, BDG, MP, LIF, OSM, TMF, PDGF, INF-α, IFN-β, and IFN-γ.

A skilled artisan recognizes that there are a variety of gene products that affect expression of nor1 and/or nur77 expression and, in some embodiments, they are utilized in the present invention. For example, the af1R gene activates the transcription of nor-1 (Chang et al., 1993).

In one embodiment of the present invention, there is a method of screening for a compound for the treatment of leukemia by providing a first vector comprising a nor-1 or nur77 nucleic acid sequence encoding a respective nor-1 or nur77 gene product, wherein the expression of said nor-1 or nur77 nucleic acid sequence is under the control of a first regulatory sequence; providing a second vector comprising a reporter nucleic acid sequence encoding a reporter gene product, wherein the expression of said reporter nucleic acid sequence is under the control of a second regulatory sequence, wherein the second regulatory sequence is responsive to nor-1 or nur77; providing a test agent; providing a leukemia cell line, wherein cells in said cell line comprise conditions suitable for expression of said nor-1 or nur77 gene product and said reporter gene product; and assaying transcriptional regulation activity of said nor-1 or nur77 gene product by measuring expression or activity of the reporter gene product in the presence of said test agent, wherein when the expression or activity of the reporter gene product changes in the presence of the test agent, the test agent is the compound for the treatment of leukemia.

A skilled artisan recognizes that the leukemic cell line may be any leukemic cell line, although exemplary leukemic cell lines include K562, U937, AML-193, HL-60, LSTRA, or CEM. In a specific embodiment, the first vector, second vector, test agent, or a combination thereof are introduced into the cell line. In another specific embodiment, the reporter nucleic acid is β-galactosidase, green fluorescent protein, blue fluorescent protein, or chloramphenicol acetyltransferase, although these are only a few exemplary embodiments and one of skill in the art would know of additional reporter nucleic acid sequences to utilize. In some embodiments, the expression or activity of the reporter gene product increases in the presence of the test agent, although in other embodiments it decreases.

In some embodiments, it is envisioned that a DNA or RNA segment comprises a nucleic acid sequence to be expressed operatively linked to its associated control sequences or an appropriate alternative. For example, the nucleic acid sequence may be operatively linked to a suitable promoter and a suitable terminator sequence. A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

The construction of such gene/control sequence DNA constructs is well-known within the art. In particular embodiments, the promoter is CMV. In certain embodiments for introduction, the DNA segment may be located on a vector, for example, a plasmid vector or a viral vector. The virus vector may be, for example, selected from the group comprising retrovirus, adenovirus, herpesvirus, vaccina virus, and adeno-associated virus. Such a DNA segment may be used in a variety of methods related to the invention. The vector may be used to deliver a particular nucleic acid sequence to a cell in a gene transfer embodiment of the invention. Also, such vectors can be used to transform cultured cells, and such cultured cells could be used, inter alia, for the expression of a particular sequence in vitro.

For a method described herein wherein a regulatory sequence responsive to nor-1, nur77, or both is utilized, a skilled artisan recognizes how to obtain the sequence by standard means in the art (see, for example, Philips et al., 1997). In particular embodiments, the regulatory sequence responsive to nor-1, nur77, or both comprises NBRE (AAAGGTCA). In other embodiments, the regulatory sequence comprises NurRE (Philips et al., 1997), or GTGATATTTACCTCCAAATGCCAG (SEQ ID NO: 36). The regulatory sequence responsive to nor-1, nur77, or both may be directly or indirectly responsive. That is, nor-1 and/or nur77 may interact with another gene product prior to interacting with the regulatory sequence. In alternative embodiments, nor-1 and/or nur77 interact with the regulatory sequence or direct the activity of another gene product to do so.

In the present invention, there is a method of identifying a compound for the treatment of leukemia by obtaining a compound suspected of having activity of a nor-1 and/or nur77 nuclear receptor agonist and determining whether the compound has the activity. For example, a compound suspected of having activity of a nor-1 and/or nur77 agonist may be a compound present in a pathway in which nor-1 and/or nur77 are also members. In a specific embodiment, the agonist is a ligand of a nor-1 and/or nur77 nuclear receptor. In another specific embodiment, the method further comprises dispersing the compound in a pharmaceutical carrier; and administering a therapeutically effective amount of the compound in the carrier to an individual having leukemia.

In an additional embodiment of the present invention, there is a mouse model for leukemia, comprising a mouse having defective nor-1 and/or nur77 nucleic acid sequences. The nucleic acid sequence(s) may be rendered defective by any standard means in the art, but in a specific embodiment the mouse is further defined as having a knockout mutation in the genes encoding nor-1 and/or nur77, respectively. The term “knockout” as used herein refers to an alteration in a coding sequence which renders the gene or gene product encoded by the coding sequence defective, such as not being expressed. The means to effect a knockout in a particular gene or nucleic acid sequence are well known in the art. In another specific embodiment, the mouse is further defined as having the nor-1KO/nur77± genotype, the nor-1±/nur77 KO, or the nor-1KO/nur77KO, wherein KO is defined as a knockout. In a further specific embodiment, the mouse comprises at least one symptom of leukemia, described elsewhere herein.

Screening Assays—Amino Acid Agonists

In a specific embodiment of the present invention there is a method of administering an agonist to nor-1 and/or nur77 nuclear receptor polypeptide. A skilled artisan recognizes that the agonist in one embodiment is a nor-1 and/or nur77 nuclear receptor ligand and enhances nor-1 and/or nur77 nuclear receptor transcriptional activity by binding to nor-1 and/or nur77 nuclear receptors. In another embodiment, the agonist is a non-ligand agonist. In some embodiments, the non-ligand agonist results in increased activity of nor-1 and/or nur77 nuclear receptor. A skilled artisan is aware that standard methods are utilized to screen for compounds that act as an agonist to nor-1 and/or nur77 nuclear receptor. For example, compound banks or oligopeptide libraries are screened in a specific embodiment by methods well known in the art for activity modulating nor-1 and/or nur77 nuclear receptor, such as its transcriptional activation activity.

One embodiment of the present invention is a method to administer compounds that affect nor-1 and/or nur77 nuclear receptor structure. Such compounds may include but are not limited to proteins, peptides, nucleic acids, carbohydrates, or other molecules, which upon binding alter nor-1 and/or nur77 nuclear receptor structure, thereby enhancing, facilitating, or increasing its activity.

One embodiment of the present invention is a method to administer a compound or compounds that affects nor-1 and/or nur77 nuclear receptor function. Such compounds may include but are not limited to proteins, nucleic acids, carbohydrates, or other molecules that upon binding (or administration if a non-ligand agonist) to improve a function of nor-1 and/or nur77 nuclear receptor.

Screening Assays—Nucleic Acid Agonists

In an embodiment of the present invention there is a method to increase nucleic acid levels of nor-1 and/or nur77 nuclear receptor. An example presented herein provides a substance that is a candidate for screening methods that are based upon whole cell assays, in vivo analysis or transformed or immortal cell lines in which a reporter gene is employed to confer on its recombinant host(s) a readily detectable phenotype that emerges only under conditions where nor-1 and/or nur77 nuclear receptor would have altered levels of its expression (such as increased). As an example, reporter genes encode a polypeptide not otherwise produced by the host cell that is detectable by analysis, e.g., by chromogenic, fluorometric, radioisotopic or spectrophotometric analysis. In a specific embodiment, at least part of nor-1 and/or nur77 nuclear receptor polynucleotide that encodes the amino acid sequence has been replaced with β-galactosidase, GFP, and the like.

Another example of a screening assay of the present invention is presented herein. Nor-1 and/or nur77 nuclear receptor-expressing cells are grown in microtiter wells, followed by addition of serial molar proportions of a candidate to a series of wells, and determination of the signal level after an incubation period that is sufficient to demonstrate expression in controls incubated solely with the vehicle that was used to resuspend or dissolve the compound. The wells containing varying proportions of candidate are then evaluated for signal activation. Candidates that demonstrate a dose-related increase of reporter gene transcription or expression are then selected for further evaluation as clinical therapeutic agents for leukemia.

In an alternative embodiment there is a method for increasing nor-1 and/or nur77 nuclear receptor polynucleotide levels by transfecting cells with nor-1 and/or nur77 nuclear receptor polynucleotide. Delivery systems for tranfection of nucleic acids into cells may utilize either viral or non-viral methods. A skilled artisan recognizes that a targeted system for non-viral forms of DNA or RNA preferably utilizes four components: 1) the DNA or RNA of interest; 2) a moiety that recognizes and binds to a cell surface receptor or antigen; 3) a DNA binding moiety; and 4) a lytic moiety that enables the transport of the complex from the cell surface to the cytoplasm. Further, liposomes and cationic lipids can be used to deliver the therapeutic gene combinations to achieve the same effect. Potential viral vectors include expression vectors derived from viruses such as adenovirus, vaccinia virus, herpes virus, and bovine papilloma virus. In addition, episomal vectors may be employed. Other DNA vectors and transporter systems are known in the art.

One skilled in the art recognizes that expression vectors derived from retroviruses, adenovirus, herpes or vaccinia viruses, or from various bacterial plasmids, may be used for delivery of nucleotides sequences to a targeted organ, tissue or cell population. Methods which are well known to those skilled in the art can be used to construct recombinant vectors which will express nor-1 and/or nur77 nuclear receptor polynucleotides.

In a specific embodiment, the transfection of nucleic acid is facilitated by a transport protein, as described in Subramanian et al. (1999). Briefly, a peptide M9 is chemically bound to a cationic peptide as a carrier molecule. The cationic complex binds the negatively charged nucleic acid of interest, followed by binding of M9 to a nuclear transport protein, such as transportin.

In a specific embodiment, there is a method of treating an organism with leukemia comprising administering therapeutically effective levels to the organism of an amino acid or nucleic acid sequence of nor-1 and/or nur77 nuclear receptor.

In another embodiment, there is a method of preventing leukemia in an organism comprising the step of increasing levels of nor-1 and/or nur77 nuclear receptor nucleic acid or amino acid sequence. The administration can be to organisms that show no signs of the onset of the disease or have early signs of the disease. In a preferred embodiment, the organism is susceptible to the leukemia or shows a genetic predisposition to having leukemia.

In a preferred embodiment, the organism described herein to be treated or subject to preventative methods is a mammal, such as a human.

The methods and treatments described herein are directed to leukemia. In a specific embodiment, the disease is systemic, and therapies would be administered to patients systemically. However, in an alternative embodiment the therapies may be administered by direct application, such as into the bone marrow.

Dosage and Formulation

The compounds (active ingredients) of this invention can be formulated and administered to treat leukemia by any means that produces contact of the active ingredient with the agent's site of action in the body of a mammal. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

The dosage administered will be a therapeutically effective amount of active ingredient and will, of course, vary depending upon known factors such as the pharmacodynamic characteristics of the particular active ingredient and its mode and route of administration; age, sex, health and weight of the recipient; nature and extent of symptoms; kind of concurrent treatment, frequency of treatment and the effect desired.

The active ingredient can be administered orally in solid dosage forms such as capsules, tablets and powders, or in liquid dosage forms such as elixirs, syrups, emulsions and suspensions. The active ingredient can also be formulated for administration parenterally by injection, rapid infusion, nasopharyngeal absorption or dernoabsorption. The agent may be administered intramuscularly, intravenously, subcutaneously, transdermally or as a suppository. In administering a compound, the compound may be given systematically. For compounds which require avoidance of systemic effects, a preferred embodiment is intrathecal administration. In a preferred embodiment, of the invention the compound is administered interarticularly for the treatment of arthritis.

Gelatin capsules contain the active ingredient and powdered carriers such as lactose, sucrose, mannitol, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solutions for parenteral administration contain preferably a water-soluble salt of the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

Additionally, standard pharmaceutical methods can be employed to control the duration of action. These are well known in the art and include control release preparations and can include appropriate macromolecules, for example polymers, polyesters, polyamino acids, polyvinyl, pyrrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Useful pharmaceutical dosage forms for administration of the compounds of this invention can be illustrated as follows. Pharmacological ranges for the active ingredients can be determined by the skilled artisan using methods well known in the art. Example ranges for active ingredients are as follows: folate ranges between 400 micrograms and 4 milligrams/day; methionine ranges between 250 mg(total) and as high as 100 mg/kg/day daily, up to 2-3 g; choline ranges between 100 mg and 2 grams; Vitamin B12 at approximately 100 micrograms orally or 1 mg intramuscularly per month; betaine ranges up to 6 grams per day; zinc ranges between 25 and 50 mg; and sodium phenylbutyrate ranges up to 20 grams per day.

Capsules: Capsules are prepared by filling standard two-piece hard gelatin capsulates each with powdered active ingredient, 175 milligrams of lactose, 24 milligrams of talc and 6 milligrams magnesium stearate.

Soft Gelatin Capsules: A mixture of active ingredient in soybean oil is prepared and injected by means of a positive displacement pump into gelatin to form soft gelatin capsules containing the active ingredient. The capsules are then washed and dried.

Tablets: Tablets are prepared by conventional procedures so that the dosage unit contains the suggested amount of active ingredient, 0.2 milligrams of colloidal silicon dioxide, 5 milligrams of magnesium stearate, 275 milligrams of microcrystalline cellulose, 11 milligrams of cornstarch and 98.8 milligrams of lactose. Appropriate coatings may be applied to increase palatability or to delay absorption.

Injectable: A parenteral composition suitable for administration by injection is prepared by stirring 1.5% by weight of active ingredients in 10% by volume propylene glycol and water. The solution is made isotonic with sodium chloride and sterilized.

Suspension: An aqueous suspension is prepared for oral administration so that each 5 milliliters contains the suggested amount of finely divided active ingredient, 200 milligrams of sodium carboxymethyl cellulose, 5 milligrams of sodium benzoate, 1.0 grams of sorbitol solution U.S. Pat. No. and 0.025 milliliters of vanillin.

Accordingly, the pharmaceutical composition of the present invention may be delivered via various routes and to various sites in an animal body to achieve a particular effect. One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The composition of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and animal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

In a specific embodiment, a drug may be transported to a target by utilizing carbonic anhydrase inhibitor (CAI) which contains a polar group such as a carboxyl group, as described in Kehayova et al., 1999. The carboxyl group renders the composition dissolvable in water, however, upon exposure to light the bond linking the CAI to the carboxyl mask breaks, allowing the remaining portion to be soluble in a hydrophobic environment.

In certain embodiments, the use of lipid formulations and/or nanocapsules is contemplated for the introduction of, for example, an agonist to nor-1 and/or nur77 nuclear receptor, a polypeptide comprising nor-1 and/or nur77 nuclear receptor amino acid sequence, a nucleic acid comprising nor-1 and/or nur77 nuclear receptor, or pharmaceutically acceptable salts thereof, polypeptides, peptides and/or agents, and/or gene therapy vectors, including both wild-type and/or antisense vectors, into host cells.

Nanocapsules can generally entrap compounds in a stable and/or reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and/or such particles may be easily made.

In a preferred embodiment, of the invention, the pharmaceutical composition may be associated with a lipid. The pharmaceutical composition associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. The lipid or lipid/pharmaceutical composition associated compositions of the present invention are not limited to any particular structure in solution. For example, they may be present in a bilayer structure, as micelles, or with a “collapsed” structure. They may also simply be interspersed in a solution, possibly forming aggregates that are not uniform in either size or shape.

Lipids are fatty substances that may be naturally occurring or synthetic lipids. For example, lipids include the fatty droplets that naturally occur in the cytoplasm as well as the class of compounds that are well-known to those of skill in the art which contain long-chain aliphatic hydrocarbons and their derivatives, such as fatty acids, alcohols, amines, amino alcohols, and aldehydes.

Phospholipids may be used for preparing the liposomes according to the present invention and may carry a net positive, negative, or neutral charge. Diacetyl phosphate can be employed to confer a negative charge on the liposomes, and stearylamine can be used to confer a positive charge on the liposomes. The liposomes can be made of one or more phospholipids.

A neutrally charged lipid can comprise a lipid with no charge, a substantially uncharged lipid, or a lipid mixture with equal number of positive and negative charges. Suitable phospholipids include phosphatidyl cholines and others that are well known to those of skill in the art.

Lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Preferably, chloroform is used as the only solvent since it is more readily evaporated than methanol.

Phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition, because of the instability and leakiness of the resulting liposomes.

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

Phospholipids can form a variety of structures other than liposomes when dispersed in water, depending on the molar ratio of lipid to water. At low ratios the liposome is the preferred structure. The physical characteristics of liposomes depend on pH, ionic strength and/or the presence of divalent cations. Liposomes can show low permeability to ionic and/or polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and/or results in an increase in permeability to ions, sugars and/or drugs.

Liposomes interact with cells via four different mechanisms: Endocytosis by phagocytic cells of the reticuloendothelial system such as macrophages and/or neutrophils; adsorption to the cell surface, either by nonspecific weak hydrophobic and/or electrostatic forces, and/or by specific interactions with cell-surface components; fusion with the plasma cell membrane by insertion of the lipid bilayer of the liposome into the plasma membrane, with simultaneous release of liposomal contents into the cytoplasm; and/or by transfer of liposomal lipids to cellular and/or subcellular membranes, and/or vice versa, without any association of the liposome contents. Varying the liposome formulation can alter which mechanism is operative, although more than one may operate at the same time.

Liposome-mediated oligonucleotide delivery and expression of foreign DNA in vitro has been very successful. Wong et al. (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the lipid may be associated with a hemagglutinating virus (HVJ). This has been shown to facilitate fusion with the cell membrane and promote cell entry of liposome-encapsulated DNA (Kaneda et al., 1989). In other embodiments, the lipid may be complexed or employed in conjunction with nuclear non-histone chromosomal proteins (HMG-1) (Kato et al., 1991). In yet further embodiments, the lipid may be complexed or employed in conjunction with both HVJ and HMG-1. In that such expression vectors have been successfully employed in transfer and expression of an oligonucleotide in vitro and in vivo, then they are applicable for the present invention. Where a bacterial promoter is employed in the DNA construct, it also will be desirable to include within the liposome an appropriate bacterial polymerase.

Liposomes used according to the present invention can be made by different methods. The size of the liposomes varies depending on the method of synthesis. A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

Liposomes within the scope of the present invention can be prepared in accordance with known laboratory techniques. In one preferred embodiment, liposomes are prepared by mixing liposomal lipids, in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In the alternative, liposomes can be prepared in accordance with other known laboratory procedures: the method of Bangham et al. (1965), the contents of which are incorporated herein by reference; the method of Gregoriadis, as described in DRUG CARRIERS IN BIOLOGY AND MEDICINE, G. Gregoriadis ed. (1979) pp. 287-341, the contents of which are incorporated herein by reference; the method of Deamer and Uster (1983), the contents of which are incorporated by reference; and the reverse-phase evaporation method as described by Szoka and Papahadjopoulos (1978). The aforementioned methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated nucleic acid is removed by centrifugation at 29,000×g and the liposomal pellets washed. The washed liposomes are resuspended at an appropriate total phospholipid concentration, e.g., about 50-200 mM. The amount of nucleic acid encapsulated can be determined in accordance with standard methods. After determination of the amount of nucleic acid encapsulated in the liposome preparation, the liposomes may be diluted to appropriate concentrations and stored at 4° C. until use.

A pharmaceutical composition comprising the liposomes will usually include a sterile, pharmaceutically acceptable carrier or diluent, such as water or saline solution.

Gene Therapy Administration

For gene therapy, a skilled artisan would be cognizant that the vector to be utilized must contain the gene of interest operatively linked to a promoter. One skilled in the art recognizes that in certain instances other sequences such as a 3′ UTR regulatory sequences are useful in expressing the gene of interest. Where appropriate, the gene therapy vectors can be formulated into preparations in solid, semisolid, liquid or gaseous forms in the ways known in the art for their respective route of administration. Means known in the art can be utilized to prevent release and absorption of the composition until it reaches the target organ or to ensure timed-release of the composition. A pharmaceutically acceptable form should be employed which does not ineffectuate the compositions of the present invention. In pharmaceutical dosage forms, the compositions can be used alone or in appropriate association, as well as in combination, with other pharmaceutically active compounds. A sufficient amount of vector containing the therapeutic nucleic acid sequence must be administered to provide a pharmacologically effective dose of the gene product.

One skilled in the art recognizes that different methods of delivery may be utilized to administer a vector into a cell. Examples include: (1) methods utilizing physical means, such as electroporation (electricity), a gene gun (physical force) or applying large volumes of a liquid (pressure); and (2) methods wherein the vector is complexed to another entity, such as a liposome or transporter molecule.

Accordingly, the present invention provides a method of transferring a therapeutic gene to a host, which comprises administering the vector of the present invention, preferably as part of a composition, using any of the aforementioned routes of administration or alternative routes known to those skilled in the art and appropriate for a particular application. Effective gene transfer of a vector to a host cell in accordance with the present invention to a host cell can be monitored in terms of a therapeutic effect (e.g. alleviation of some symptom associated with the particular disease being treated) or, further, by evidence of the transferred gene or expression of the gene within the host (e.g., using the polymerase chain reaction in conjunction with sequencing, Northern or Southern hybridizations, or transcription assays to detect the nucleic acid in host cells, or using immunoblot analysis, antibody-mediated detection, mRNA or protein half-life studies, or particularized assays to detect protein or polypeptide encoded by the transferred nucleic acid, or impacted in level or function due to such transfer).

These methods described herein are by no means all-inclusive, and further methods to suit the specific application will be apparent to the ordinary skilled artisan. Moreover, the effective amount of the compositions can be further approximated through analogy to compounds known to exert the desired effect.

Furthermore, the actual dose and schedule can vary depending on whether the compositions are administered in combination with other pharmaceutical compositions, or depending on interindividual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

It is possible that cells containing the therapeutic gene may also contain a suicide gene (i.e., a gene which encodes a product that can be used to destroy the cell, such as herpes simplex virus thymidine kinase). In many gene therapy situations, it is desirable to be able to express a gene for therapeutic purposes in a host cell but also to have the capacity to destroy the host cell once the therapy is completed, becomes uncontrollable, or does not lead to a predictable or desirable result. Thus, expression of the therapeutic gene in a host cell can be driven by a promoter although the product of the suicide gene remains harmless in the absence of a prodrug. Once the therapy is complete or no longer desired or needed, administration of a prodrug causes the suicide gene product to become lethal to the cell. Examples of suicide gene/prodrug combinations which may be used are Herpes Simplex Virus-thymidine kinase (HSV-tk) and ganciclovir, acyclovir or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

The method of cell therapy may be employed by methods known in the art wherein a cultured cell containing a non-defective nor-1 and/or nur77 nuclear receptor nucleic acid sequence encoding nor-1 and/or nur77 nuclear receptor polypeptide is introduced.

In another embodiment, biologically active molecules, such as vectors for gene therapy, are incorporated in a large hydration domain between “pinched” regions of a lipid-poly-L-glutamic acid (PGA) complex, where the PGA and the cationic lipid didodecyl dimethylammonium bromide associate to form localized pinched regions, for delivery applications (Subramaniam, et al., 2000).

In an alternative embodiment, an amino acid sequence is engineered to accumulate as an aggregate in the endoplasmic reticulum, followed by administration of a composition to induce protein disaggregation, resulting in rapid and transient secretion (Rivera et al., 2000).

A peptide (11 amino acids) derived from HIV has been recently described that when fused to full length proteins and injected into mice allow a rapid dispersal to the nucleus of all cells of the body (Schwarze et al., 1999). Schwarze et al. made fusion proteins to Tat ranging in size from 15 to 120 kDa. They documented a rapid uptake of the fusion proteins to the nuclei of cells throughout the animal, and the functional activity of the proteins was retained.

In an embodiment of the present invention there are constructs containing the Tat or Tat-HA nucleic acid sequence operatively linked to the nor-1 and/or nur77 nuclear receptor nucleic acid sequence. The vectors are expressed in bacterial cultures and the fusion protein is purified. This purified Tat-HA-nor-1/nur77 nuclear receptor protein or Tat-nor-1/nur77 nuclear receptor protein is injected into the animal to determine the efficiency of the Tat delivery system into the particular site of delivery, such as into the bone marrow, or by means to deliver the fusion protein systemically. Analysis is carried out to determine the potential of the Tat-HA-nor-1/nur77 nuclear receptor protein or Tat-nor-1/nur77 nuclear receptor protein in alleviation of any leukemia symptom. This is a viable therapeutic approach either in its own right or in association with other methods, treatments or genes.

DNA Delivery Using Viral Vectors

The ability of certain viruses to infect cells via receptor-mediated endocytosis, to integrate into host cell genome and to express viral genes stably and efficiently have made them attractive candidates for the transfer of foreign genes into mammalian cells. Preferred gene therapy vectors of the present invention will generally be viral vectors.

Although some viruses that can accept foreign genetic material are limited in the number of nucleotides they can accommodate and in the range of cells they infect, these viruses have been demonstrated to successfully effect gene expression. However, adenoviruses do not integrate their genetic material into the host genome and therefore do not require host replication for gene expression, making them ideally suited for rapid, efficient, heterologous gene expression. Techniques for preparing replication-defective infective viruses are well known in the art.

Of course, in using viral delivery systems, one will desire to purify the virion sufficiently to render it essentially free of undesirable contaminants, such as defective interfering viral particles, endotoxins, and other pyrogens such that it will not cause any untoward reactions in the cell, animal or individual receiving the vector construct. A preferred means of purifying the vector involves the use of buoyant density gradients, such as cesium chloride gradient centrifugation.

a. Adenoviral Vectors

A particular method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus vector” is meant to include those constructs containing adenovirus sequences sufficient to (a) support packaging of the construct and (b) to ultimately express a tissue or cell-specific construct that has been cloned therein.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and/or Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and/or propagation of the current adenovirus vectors, which are replication deficient, depend on a unique helper cell line, designated 293, which was transformed from embryonic kidney cells by Ad5 DNA fragments and constitutively expresses E1 proteins (E1A and/or Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). Recently, adnoviral vectors comprising deletions in the E4 region have been described (U.S. Pat. No. 5,670,488, incorporated herein by reference).

In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and/or E3 regions, the maximum capacity of the current adenovirus vector is under 7.5 kb, and/or about 15% of the total length of the vector. More than 80% of the adenovirus viral genome remains in the vector backbone.

Helper cell lines may be derived from mammalian cells such as human embryonic kidney cells, muscle cells, hematopoietic cells and other human embryonic mesenchymal or epithelial cells. Alternatively, the helper cells may be derived from the cells of other mammalian species that are permissive for adenovirus. Such cells include, e.g., Vero cells and/or other monkey embryonic mesenchymal and/or epithelial cells. As stated above, the preferred helper cell line is 293.

Recently, Racher et al. (1995) disclosed improved methods for propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and/or left stationary, with occasional agitation, for 1 to 4 h. The medium is then replaced with 50 ml of fresh medium and/or shaking initiated. For virus production, cells are allowed to grow to about 80% confluence, after which time the medium is replaced (to 25% of the final volume) and/or adenovirus added at an MOI of 0.05. Cultures are left stationary overnight, following which the volume is increased to 100% and/or shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes and subgroups A-F. Adenovirus type 5 of subgroup C is the preferred starting material in order to obtain the conditional replication-defective adenovirus vector for use in the present invention. This is because Adenovirus type 5 is a adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the NURR subfamily member may also be inserted in lieu of the deleted E3 region in E3 replacement vectors as described by Karlsson et al (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al, 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and/or Horwitz, 1992; Graham and/or Prevec, 1992). Recently, animal studies suggested that recombinant adenovirus could be used for gene therapy (Stratford-Perricaudet and/or Perricaudet, 1991a; Stratford-Perricaudet et al., 1991b; Rich et al., 1993). Studies in administering recombinant adenovirus to different tissues include trachea instillation (Rosenfeld et al., 1991; Rosenfeld et al., 1992), muscle injection (Ragot et al, 1993), peripheral intravenous injections (Herz and/or Gerard, 1993) and stereotactic inoculation into the brain (Le Gal La Salle et al., 1993). Recombinant adenovirus and adeno-associated virus (see below) can both infect and transduce non-dividing mammalian primary cells.

b. Adeno-Associated Viral Vectors

Adeno-associated virus (AAV) is an attractive vector system for use in the cell transduction of the present invention as it has a high frequency of integration, and it can infect nondividing cells, thus making it useful for delivery of genes into mammalian cells, for example, in tissue culture (Muzyczka, 1992) and in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Yoder et al., 1994; Zhou et al., 1994; Hermonat and/or Muzyczka, 1984; Tratschin et al., 1985; McLaughlin et al., 1988) or genes involved in mammalian diseases (Flotte et al., 1992; Luo et al., 1994; Ohi et al., 1990; Walsh et al., 1994; Wei et al., 1994). Recently, an AAV vector has been approved for phase I trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild type AAV genome integrates through its ends into chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild type AAV coding sequences without the terminal repeats, for example pEM45 (McCarty et al., 1991; incorporated herein by reference). The cells are also transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte et al., 1995).

C. Retroviral Vectors

Retroviruses have promise as gene delivery vectors due to their ability to integrate their genes into the host genome, transferring a large amount of foreign genetic material, infecting a broad spectrum of species and cell types and of being packaged in special cell-lines (Miller, 1992).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and/or Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, and env sequences integrated in the host cell genome. However, new packaging cell lines are now available that should greatly decrease the likelihood of recombination (Markowitz et al, 1988; Hersdorffer et al., 1990).

Gene delivery using second generation retroviral vectors has been reported. Kasahara et al. (1994) prepared an engineered variant of the Moloney murine leukemia virus, which normally infects only mouse cells, that modified an envelope protein so that the virus specifically bound to, and infected, mammalian cells bearing the erythropoietin (EPO) receptor. This was achieved by inserting a portion of the EPO sequence into an envelope protein to create a chimeric protein with a new binding specificity.

d. Other Viral Vectors

Other viral vectors may be employed as expression constructs in the present invention. Vectors derived from viruses such as vaccinia virus (Ridgeway, 1988; Baichwal and/or Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and/or Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In certain further embodiments, the gene therapy vector will be HSV. A factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations. HSV also is relatively easy to manipulate and can be grown to high titers. Thus, delivery is less of a problem, both in terms of volumes needed to attain sufficient MOI and in a lessened need for repeat dosings.

e. Modified Viruses

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein or against a specific cell receptor were used. The antibodies were coupled via the biotin components by using streptavidin (Roux et al., 1989). Using antibodies against major histocompatibility complex class I and class II antigens, they demonstrated the infection of a variety of mammalian cells that bore those surface antigens with an ecotropic virus in vitro (Roux et al., 1989).

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Pathology of Mice Deficient for Nor-1 and Nur77

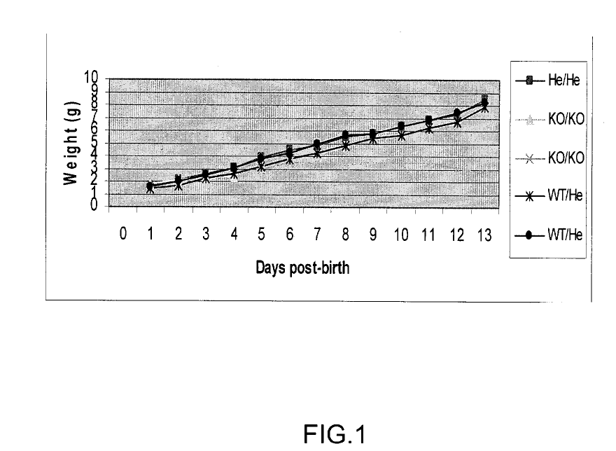

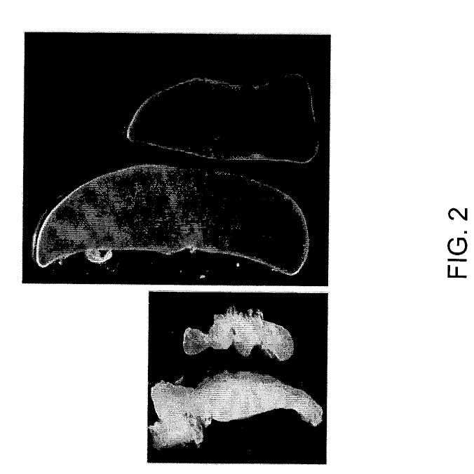

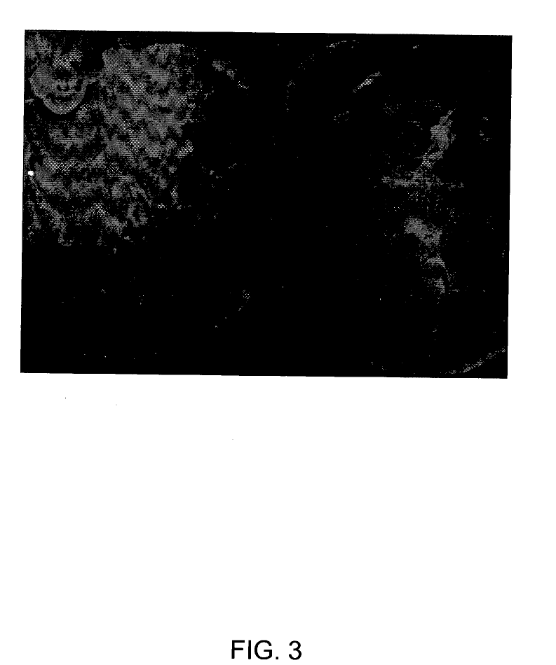

Mice deficient for both nor-1 and nur77 (nor-1KO/nur77KO) were generated. Shortly after birth, the nor-1KO/nur77KO mice begin to waste and appear lethargic. FIG. 1 shows the growth curve of one litter including two nor-1KO/nur77KO mice and their normal littermates that is representative of a number of litters that were weighed daily for a period of two weeks. After a short period of weight loss and increasing weakness, these mice become moribund and succumb to death. The postnatal day of death varies greatly in these mice, however, generally occurs prior to the fourth week of life. Upon necropsy the nor-1KO/nur77KO mice display lymphadenopathy and splenomegaly (FIG. 2). The lymph node and spleen are both lymphoid tissues and these defects suggested alteration in hematopoiesis. In addition, liver discoloration consistent with cellular infiltration was noted (FIG. 3).

Blood samples were analyzed from several nor-1KO/nur77KO mice showing disease symptoms. Table 1 shows the results obtained from two nor-1KO/nur77KO mice at varying ages and their corresponding normal littermates. Both of the double knockout mice showed elevated total white blood cells (leukocytosis).

TABLE 1
Peripheral Blood Counts at Postnatal Day 14-16

| nor-1/nur77 | WT/WT | KO/KO |
|---|---|---|
| WBC × | 2.86 ± 0.5 | 7.70 ± 0.7^{c} |
| 10 \hat{ } 3/uL^{a} | | |
| RBC × | 5.43 ± 0.3 | 3.23 ± 0.5^{c} |
| 10 \hat{ } 6/uL^{a} | | |
| HGB g/dL^{a} | 10.7 ± 0.6 | 6.17 ± 1.3^{c} |
| % HCT^{a} | 32.5 ± 1.2 | 19.5 ± 3.2^{c} |
| % Neutrophils^{b} | 23.0 ± 2.0 | 14.0 ± 5.0 |
| % Lymphocytes^{b} | 70.0 ± 2.0 | 13.0 ± 7.0^{c} |
| % Monocytes^{b} | 0 ± 0 | 0 ± 0 |
| % Eosinophils^{b} | 4.0 ± 1.0 | 0 ± 0^{c} |
| % Basophils^{b} | 0 ± 0 | 0 ± 0 |
| % Young Forms^{b} | 3.0 ± 1.0 | 84.0 ± 4.0^{c} |

WT = wildtype, KO = knock out, WBC = white blood cell, RBC = red blood cell, HGB = hemoglobin, HCT = hematocrit. Results are the mean (± SEM) of 3 mice of each genotype.
^{a}Values determined by Technicon Hematology System.
^{b}Percentage of cells determined by differential counts on at least 200 white blood cells per blood smear.
^{c}p ≦ 0.05.

Automated and manual differential analysis of the peripheral blood in the nor-1KO/nur77KO mice compared to the normal littermates showed a substantial increase in the percentage and total number of neutrophils (neutrophilia) in double knock out mice. The double knock out mice were anemic, illustrated by pale blood color, decreased number of red blood cells and decreased concentration of hemoglobin, and lower hematocrit values. Leukocytosis, neutrophilia, and anemia are classic symptoms of leukemia in both human patients and other mouse models of leukemia. An increase in young myeloid forms was also noted during differential analysis and is often observed in patients with various forms of myeloid leukemia.

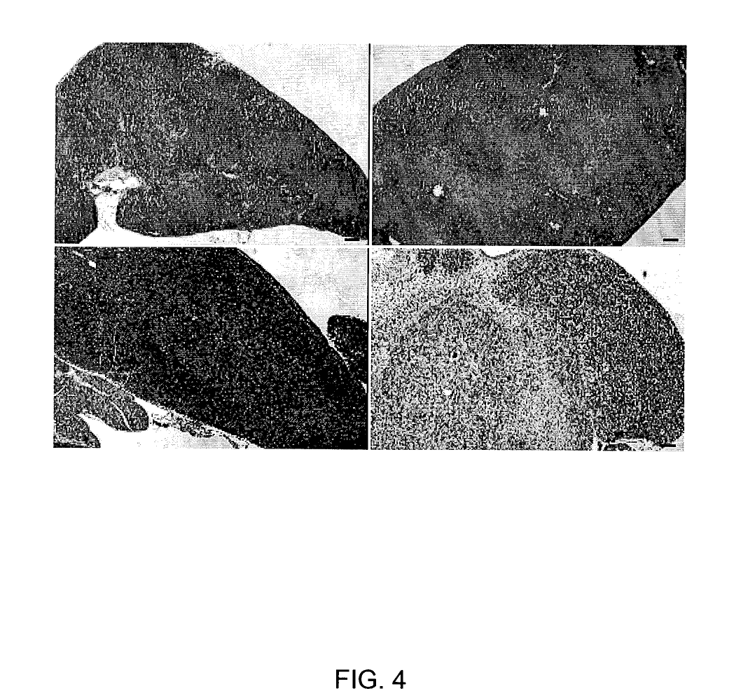

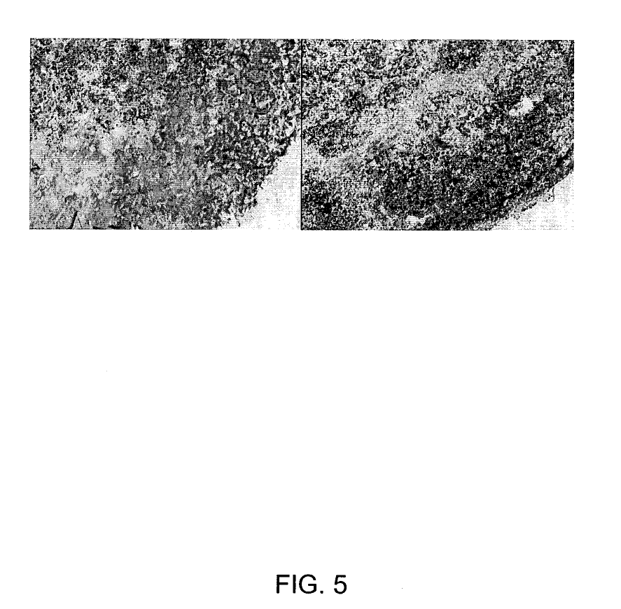

Histological examination of lymphoid tissues of the nor-1KO/nur77KO animals showed disrupted architecture (FIG. 4). Specifically, the spleen showed loss of distinct lymphocytic nodules that is seen in normal spleens. Normally, the thymus contains a distinct darkly staining cortex and a paler staining medulla. However the nor1KO/nur77KO thymus has lost the classic cortical-medullary architecture. In addition, abnormal encapsulation and tumor-like septae were present in the thymus. The pale staining appearance of the thymus suggested to us that medullary epithelial cells might be part of this abnormal histology. Immunohistochemistry using antibodies specific to medullary epithelial cells confirmed this suspicion (FIG. 5). Disrupted architecture in both the spleen and the thymus is consistent with a global hematopoietic disorder such as leukemia.

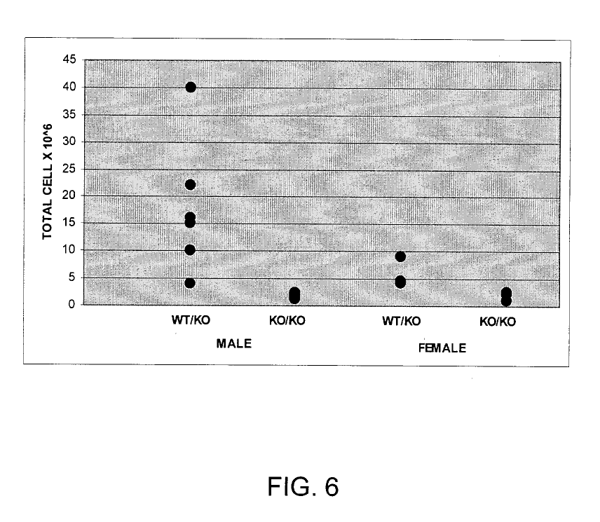

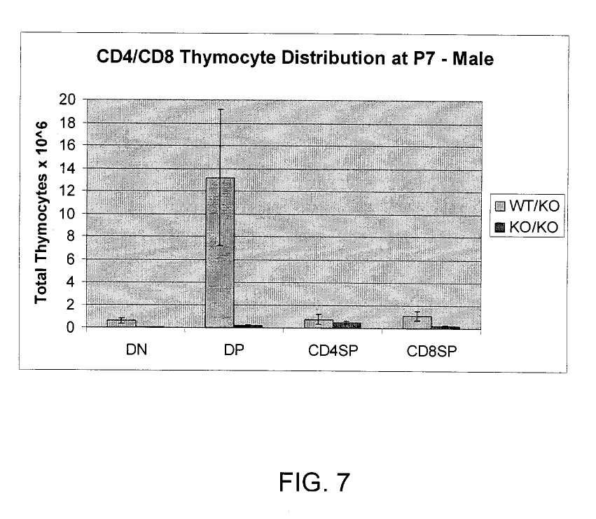

Based on previous results implicationg both nor-1 and nur77 during negative selection in the thymus, it was possible the histological defects mentioned above were due to abnormal survival of lymphocytes that would normally die during negative selection. Thymocytes were harvested from mice at postnatal day 7 and counted on a hematocytometer. In contrast to the predictions, consistently less thymocytes were obtained from the nor-1KO/nur77KO thymuses as compared to normal thymuses (FIG. 6). Even more unexpected, flow cytometric analysis of well defined developmental stages based on expression of cell surface antigens CD4 and CD8 showed that this decrease was not limited to the later stage of negative selection (FIG. 7). These data show that nor-1 and nur77 play a role in early lymphocyte development prior to negative selection within the thymus and/or the bone marrow that has never been reported before.

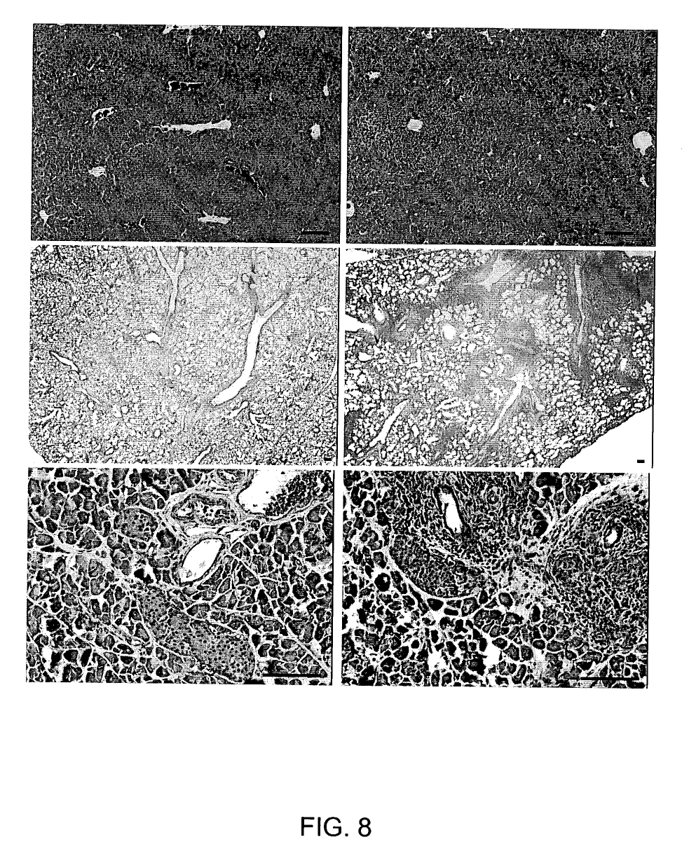

Histology of nonlymphoid tissues obtained from nor-1KO/nur77KO mice revealed extensive perivascular cellular infiltration in the lung, liver, and pancreas, tissues that are often affected in leukemic patients (FIG. 8). These infiltrates within the lung were extensive enough to speculate that the opening of the airways of these mice may be impeded which could explain the labored breathing observed in the moribund mice just prior to death.

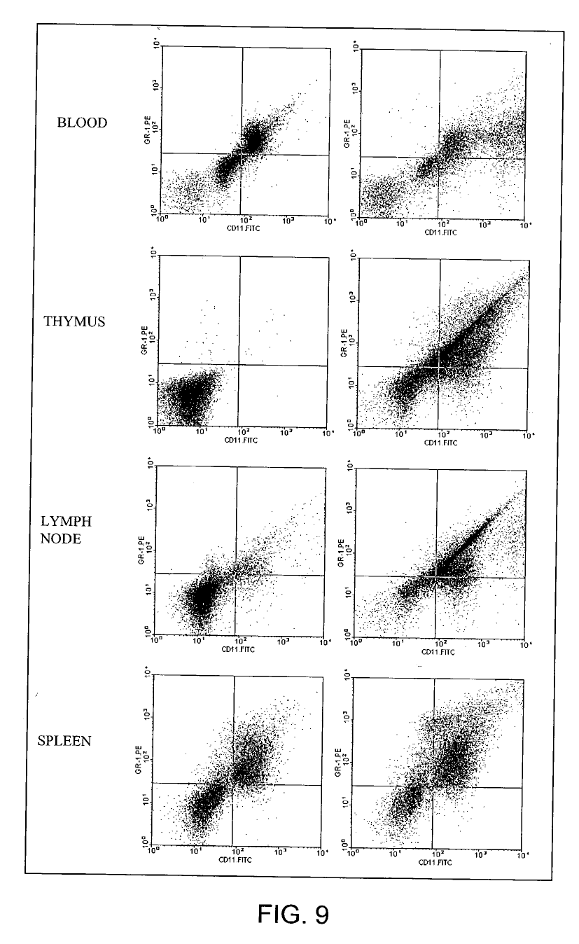

To further characterize the disrupted histology within the nor-1KO/nur77KO mice, flow cytometry was used to analyze the cell types within the lymphoid tissues and peripheral blood. Analysis of CD11b, a cell surface marker for the myeloid lineage and GR-1 a marker for granulocytes that differentiate from the myeloid lineage revealed and increase of CD11b+ and CD11b+/GR+ cells within the thymus, peripheral blood, spleen, and lymph node of diseased animals as compared to normal littermates (FIG. 9). This data is consistent with the neutrophilia found during blood analysis of the nor-1KO/nur77KO mice. CD11b and GR-1 expression are increased in blood from patients with both chronic and acute myeloid leukemia.

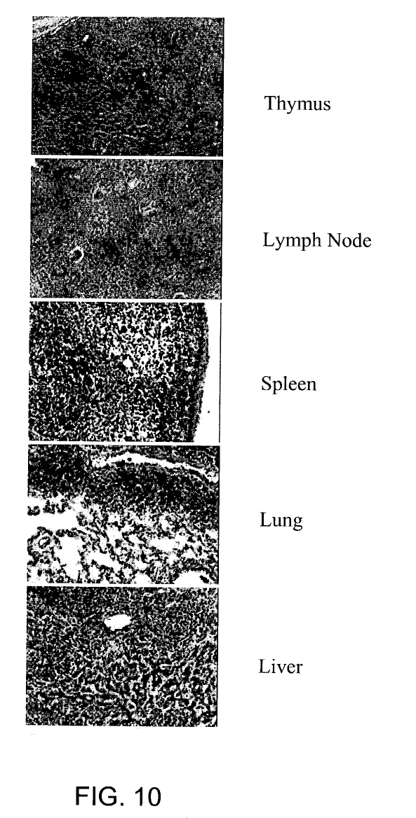

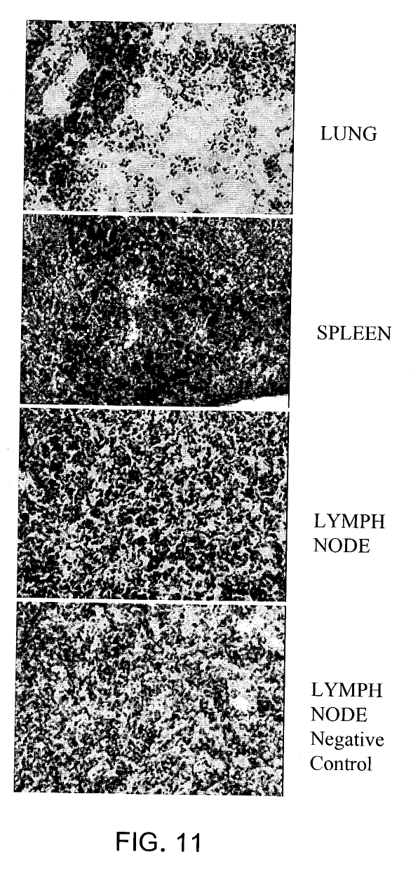

The flow cytometry data showing increased levels of CD11b+ cells and neutrophilia detected in blood analysis from the knock out mice suggested that the abnormal cellular infiltrates observed in the nonlymphoid tissues might consist of myeloid cells. This was confirmed by staining histological tissue sections of the cellular infiltrates with myeloperoxidase, a stain often used to identify cells of myeloid origin in tissue samples and blood obtained from patients with myeloid leukemia (FIG. 10). The presence of leukemic myeloid cells within both the lymphoid and nonlymphoid tissues was further confirmed with immunohistochemistry using an antibody that binds to the CD11b molecule on the cell surfaces (FIG. 11).

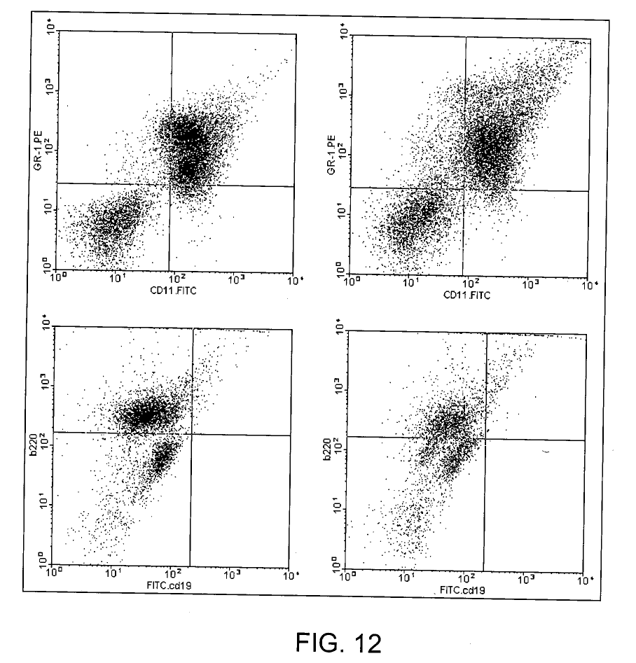

The above data lead to the diagnosis of myeloid leukemia with differentiation similar to that of human chronic myeloid leukemia in diseased nor-1KO/nur77KO mice. Leukemia is a result of a primary defect of hematopoiesis within the bone marrow. Therefore, the bone marrow of nor-1KO/nur77KO mice was examined using flow cytometry. It has been reported that while leukemia begins within the bone marrow, changes in cell composition sometimes aren't detected due to the rapid exit of the abnormal cells into the periphery although this is not always the case in well developed leukemia. The analysis revealed an increase in the percentage of CD11b+ myeloid cells (FIG. 12). In addition, decreased percentages of B220+ cells of the b-lymphocyte population were observed. A shift in hematopoiesis to production of CD11b+ cells and a decrease in other hematopoietic lineages is consistent with what is observed in both acute and chronic leukemia. These results also confirm and essential role of nor-1 and nur77 in regulation of bone marrow hematopoiesis and prevention of the development of leukemia.

### Example 2

Pathology of Hypoallelic Nor-1 and Nur77 Deficient Mice

While breeding to obtain nor-1KO/nur77KO mice, mice with one allele of either gene remaining, nor-1±/nur77KO or nor-1KO/nur77± mice, were generated. These mice approximately contain one quarter of the normal level of total nor-1 and nur77 protein. These mice are referred to herein as hypoallelic.

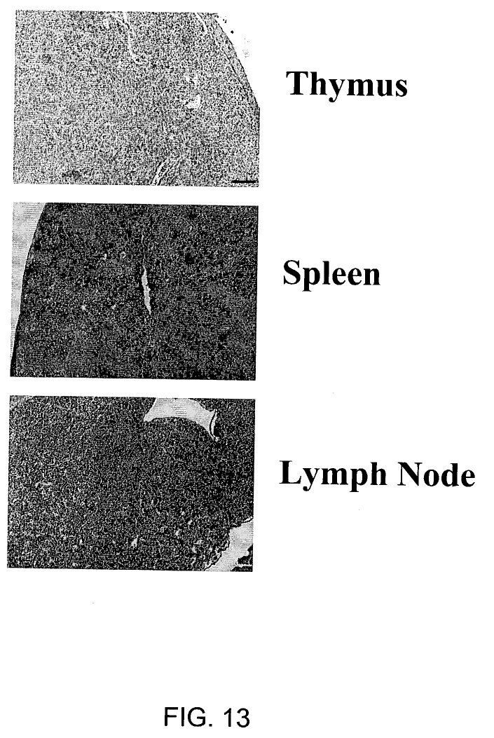

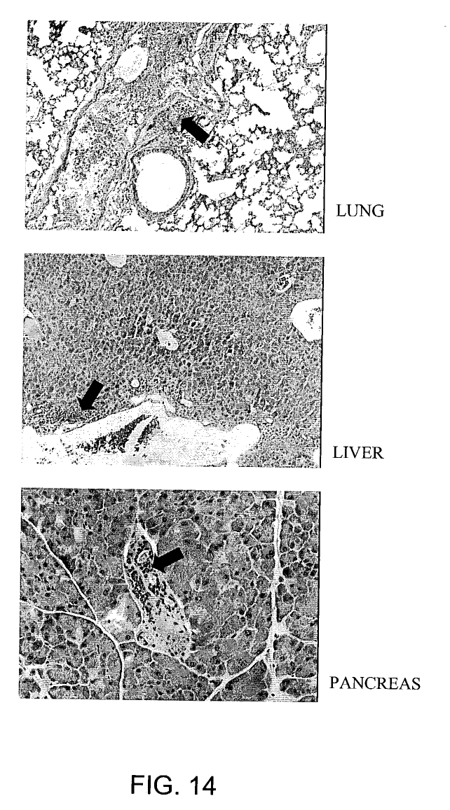

Initially these mice appear normal. However, by 3-4 months of age, they begin to show similar outward signs of disease as the nor-1KO/nur77KO mice. Upon necropsy of the diseased animals, splenomegaly, lymphadenopathy, and discoloration of the liver was noted. Histological examination of the lymphoid tissue revealed a similar phenotype as the nor-1KO/nur77KO (FIG. 13). The normal cortical/medullary junction of the thymus has been lost in these hypoallelic mice. The spleen of these mice has lost distinct nodular architecture. In addition, like the nor-1KO/nur77KO mice, extensive perivascular cellular infiltrates were noted in the liver, lung, and pancreas (FIG. 14). To further confirm that the hypoallelic mice were also developing myeloid leukemia, blood analysis was performed (Table 2).

TABLE 2
Peripheral Blood Analysis of Hypoallelic mice in comparison to normal littermate.

| | AGE (months) | WBC × 10³/ul | RBC × 10⁶/ul | HGB g/dL | HCT % | % Neutrophils | % LUC |
|---|---|---|---|---|---|---|---|
| KO/HE | 5 | 4.75 | 7.24 | 11.7 | 33 | 61.1 | 2 |
| HE/KO | 3 | 6.5 | 9.22 | 13.7 | 43.5 | 23.5 | 2.7 |
| WT/KO | 3 | 1.25 | 9.2 | 14.5 | 45.5 | 8.8 | 0.9 |

WBC = white blood cell; RBC = red blood cell; HGB = hemoglobin; HCT = hematocrit; LUC = large unstained cells; WT = wildtype; HE = heterozygous, +/− (one allele); KO = knock out, −/−, homozygous (no alleles)

Just as in the nor-1KO/nur77KO animals, the hypoallelic animals showed leukocytosis, neutrophilia, anemia, and an increase in the percentage of large unstained cells as compared to normal levels. Large unstained cells are often increased in automated count values when young hematopoietic cells are present in the circulation. The hypoallelic mice also develop myeloid leukemia, however, at a later age. This difference shows that even at one-quarter of their normal level and activity, nor-1 or nur77 can maintain regulated myeloid hematopoiesis and prevent the onset of leukemia for a period of time. Thus, the level of expression or activity of nor-1 or nur77 is critical to protection against the development of leukemia.

Thus, rapidly developing myeloid leukemia in the absence of nor-1 and nur77 show that these genes are critical during hematopoiesis. No reports have ever implicated nor-1, nur77, or their relative nurr1 during this process. In addition, the hypoallelic mouse model provided herein that retains only one-quarter of the normal level and activity of nor-1 or nur77 also develops leukemia, although at a later stage. This illustrates how crucial the amount and activity of these nuclear receptors is during hematopoiesis. In specific embodiments, increasing either the level or activity of nor-1 or nur77 prevents against the onset of or alters the progression of unregulated proliferation of hematopoietic cells that occurs during myeloid leukemia.

References

All patents and publications mentioned in the specification are indicative of the levels of those skilled in the art to which the invention pertains. All patents and publications are herein incorporated by reference to the same extent as if each individual publication was specifically and individually indicated to be incorporated by reference.

Patents

U.S. Pat. No. 4,797,368

U.S. Pat. No. 5,139,941

U.S. Pat. No. 5,670,488

U.S. Pat. No. 6,541,217

Publications

Bandoh, S. et al. Mechanical agitation induces gene expression of NOR-1 and its closely related orphan nuclear receptors in leukemic cell lines. Leukemia (1997) 11:1453-1458.

Chang, P. K., Cary, J. W., Bhatnagar, D., Cleveland, T. E., Bennett, J. W., Linz, J. E., Woloshuk, C. P. and Payne, G. A. Cloning of the Aspergillus parasiticus apa-2 gene associated with the regulation of aflatoxin biosynthesis. Appl. Environ. Microbiol. 59 (10), 3273-3279 (1993).

Wu W. -S. et al. Promyelocytic leukemia protein PML inhibits Nur77-mediated transcription through specific functional interactions. Oncogene (2002) 21: 3925-3933.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of inhibiting proliferation of a hematopoietic cell, comprising the step of modulating the level of nor-1 and/or nur77 nuclear receptor.

2. The method of claim 1, wherein the hematopoietic cell is a hematopoietic stem cell.

3. The method of claim 1, wherein the hematopoietic cell is a hematopoietic myeloid cell.

4. The method of claim 1, wherein the modulating step is defined as increasing the level of a nor-1 and/or nur77 nuclear receptor polypeptide.

5. The method of claim 4, wherein the increasing step is defined as increasing the level of a nor-1 and/or nur77 nuclear receptor polynucleotide.

6. The method of claim 5, wherein the nor-1 and/or nur77 nuclear receptor polynucleotide is increased through administration of a vector comprising the polynucleotide.

7. The method of claim 6, wherein the vector is a viral vector or a non-viral vector.

8. The method of claim 7, wherein the viral vector is an adenoviral vector, a retroviral vector, or an adeno-associated vector.

9. The method of claim 7, wherein the viral vector is an adenoviral vector.

10. The method of claim 7, wherein the non-viral vector is a plasmid.

11. The method of claim 5, wherein the nor-1 and/or nur77 nuclear receptor polynucleotide is increased through upregulation of expression.

12. The method of claim 11, wherein the upregulation of expression is of the nor-1 and/or nur77 nuclear receptor.

13. The method of claim 12, wherein the upregulation of expression of the nor-1 and/or nur77 nuclear receptor is through administration of growth factors, cytokines, cyclic AMP, or a mixture thereof.

14. The method of claim 1, wherein the cell is in a mammal afflicted with leukemia.

15. A method of inhibiting proliferation of a hematopoietic cell, comprising the step of modulating the activity of a nor-1 and/or nur77 nuclear receptor.

16. The method of claim 15, wherein the hematopoietic cell is a hematopoietic stem cell.

17. The method of claim 15, wherein the hematopoietic cell is a hematopoietic myeloid cell.

18. The method of claim 15, wherein the modulating step is defined as increasing transcriptional activity of a nor-1 and/or nur77 nuclear receptor polypeptide.

19. The method of claim 15, wherein the modulating step is further defined as administering an agonist to the nor-1 and/or nur77 nuclear receptor polypeptide.

20. A method of treating leukemia in an individual, comprising the step of modulating a nor-1 and/or nur77 nuclear receptor in said individual.

21. The method of claim 20, wherein said modulating step occurs in a hematopoietic cell of the individual.

22. The method of claim 21, wherein the hematopoietic cell is a hematopoietic stem cell.

23. The method of claim 21, wherein the hematopoietic cell is a hematopoietic myeloid cell.

24. The method of claim 20, wherein the modulating step is further defined as increasing the activity of a nor-1 and/or nur77 nuclear receptor polypeptide.

25. The method of claim 20, wherein the modulating step is further defined as increasing the level of a nor-1 and/or nur77 nuclear receptor polypeptide.

26. The method of claim 20, wherein the modulating step is further defined as increasing the level of a nor-1 and/or nur77 nuclear receptor polynucleotide.

27. The method of claim 24, wherein the increasing activity step is further defined as introducing an agonist to said nor-1 and/or nur77 nuclear receptor polypeptide.

28. The method of claim 27, wherein the introducing step is further defined as administering said agonist in a pharmaceutically acceptable composition to said individual.

29. The method of claim 27, wherein the agonist is a ligand of said nor-1 and/or nur77 nuclear receptor.

30. The method of claim 27, wherein the agonist is not a ligand of said nor-1 and/or nur77 nuclear receptor.

31. The method of claim 26, wherein the increasing the level of a nor-1 and/or nur77 nuclear receptor polynucleotide step is defined as increasing expression of a respective nor-1 and/or nur77 nuclear receptor in a cell of the individual.

32. The method of claim 31, wherein the cell is a hematopoietic bone marrow stem cell.

33. The method of claim 31, wherein the cell is a hematopoietic myeloid cell.

34. The method of claim 26, wherein the increasing the level of a nor-1 and/or nur77 nuclear receptor polynucleotide step is defined as increasing the half-life of a respective nor-1 and/or nur77 nuclear receptor mRNA in a cell of the individual.

35. The method of claim 34, wherein the cell is a hematopoietic bone marrow stem cell.

36. The method of claim 34, wherein the cell is a hematopoietic myeloid cell.

37. The method of claim 32, wherein the method further comprises the step of administering said cell to an individual.

38. The method of claim 33, wherein the method further comprises the step of administering said cell to an individual.

39. The method of claim 35, wherein the method further comprises the step of administering said cell to an individual.

40. The method of claim 36, wherein the method further comprises the step of administering said cell to an individual.

41. A method of increasing the level of a nor-1 and/or nur77 nuclear receptor in a hematopoietic cell, comprising the step of administering a compound to the cell to increase the expression of said nor-1 and/or nur77 nuclear receptor.

42. The method of claim 41, wherein said compound is a growth factor, cytokine, cyclic AMP, or a mixture thereof.

43. The method of claim 41, wherein said method is further defined as administering said compound in a pharmaceutically acceptable composition to said individual.

44. A method of identifying an upregulator of expression of a nor-1 and/or nur77 nuclear receptor, comprising the steps of:
introducing to a cell a test agent, wherein the cell comprises a marker sequence and wherein the expression of the marker sequence is regulated by a nor-1 and/or nur77 nuclear receptor regulatory sequence; and
measuring for an increase in the expression level of the marker sequence, wherein when said increase occurs following introduction of said test agent to said cell, said test agent is said upregulator.

45. The method of claim 44, wherein the method further comprises administering the upregulator in a pharmaceutically acceptable composition to an individual.

46. The method of claim 45, wherein the individual is susceptible to leukemia or is diagnosed with leukemia.

47. A method of identifying a compound for the treatment of leukemia, comprising the steps of:
obtaining a compound suspected of having activity of a nor-1 and/or nur77 nuclear receptor agonist; and
determining whether said compound has said activity.

48. The method of claim 47, wherein the agonist is a ligand of a nor-1 and/or nur77 nuclear receptor.

49. The method of claim 47, wherein the method further comprises:
dispersing the compound in a pharmaceutical carrier; and
administering a therapeutically effective amount of the compound in the carrier to an individual having leukemia.

50. As a composition of matter, the compound obtained by the method of claim 47.

51. A pharmacologically acceptable composition comprising:
the compound obtained by the method of claim 47; and
a pharmaceutical carrier.

52. A method of screening for a compound for the treatment of leukemia, comprising the steps of:
providing a first vector comprising a nor-1 or nur77 nucleic acid sequence encoding a respective nor-1 or nur77 gene product, wherein the expression of said nor-1 or nur77 nucleic acid sequence is under the control of a first regulatory sequence;
providing a second vector comprising a reporter nucleic acid sequence encoding a reporter gene product, wherein the expression of said reporter nucleic acid sequence is under the control of a second regulatory sequence, wherein the second regulatory sequence is responsive to nor-1 or nur77;
providing a test agent;
providing a leukemia cell line, wherein cells in said cell line comprise conditions suitable for expression of said nor-1 or nur77 gene product and said reporter gene product; and
assaying transcriptional regulation activity of said nor-1 or nur77 gene product by measuring expression or activity of the reporter gene product in the presence of said test agent, wherein when the expression or activity of the reporter gene product changes in the presence of the test agent, the test agent is the compound for the treatment of leukemia.

53. The method of claim 52, wherein the leukemic cell line is K562, U937, AML-193, HL-60, LSTRA, or CEM.

54. The method of claim 52, wherein the first vector, second vector, test agent, or a combination thereof are introduced into the cell line.

55. The method of claim 52, wherein the reporter nucleic acid is β-galactosidase, green fluorescent protein, blue fluorescent protein, or chloramphenicol acetyltransferase.

56. The method of claim 52, wherein the expression or activity of the reporter gene product increases in the presence of the test agent.

57. A mouse model for leukemia, comprising a mouse having defective nor-1 and nur77 nucleic acid sequences.

58. The mouse model of claim 57, wherein the mouse is further defined as having a knockout mutation in the genes encoding nor-1 and nur77, respectively.

59. The mouse model of claim 57, wherein the mouse is further defined as having the nor-1KO/nur77± genotype, the nor-1±/nur77 KO, or the nor-1KO/nur77KO, wherein KO is defined as a knockout.

60. The mouse model of claim 57, wherein the mouse comprises at least one symptom of leukemia.


SEQUENCE LISTING (36 sequences)

SEQ ID NO: 1 - DNA, 3802 bp, Homo sapiens
ataaatgacgtgccgagagagcgagcgaacgcgcagccgggagagcggagtctcctgcctcccgccccccacccctccagctcctgctcctcctccgctccccatacacagacgcgctcacacccgctccctcactcgaacacacagacacaagcgcgcacacaggctccgcacacacacacttcgctctcccgcgcgctcacacccctcttgccctgagcccttgccggtgcagcgcggcgccgcagctggacgcccctcccgggctcactttgcaacgctgacggtgccggcagtggccgtggaggtgggaacagcggcggcatcctcccccctggtcacagcccaagccaggacgcccgcggaacctctcggctgtgctctcccatgagtcgggatcgcagcatcccccaccagccgctcaccgcctccgggagccgctgggcttgtacaccgcagcccttccgggacagcagctgtgactcccccccagtgcagatttcgggacagctctctagaaactcgctctaaagacggaaccgccacagcactcaaagcccactgcggaagagggcagcccggcaagcccgggccctgagcctggacccttagcggtgccgggcagcactgccggcgcttcgcctcgccggacgtccgctcctcctacactctcagcctccgctggagagacccccagccccaccattcagcgcgcaagataccctccagatatgccctgcgtccaagcccaatatagcccttcccctccaggttccagttatgcggcgcagacatacagctcggaatacaccacggagatcatgaaccccgactacaccaagctgaccatggaccttggcagcactgagatcacggctacagccaccacgtccctgcccagcatcagtaccttcgtggagggctactcgagcaactacgaactcaagccttcctgcgtgtaccaaatgcagcggcccttgatcaaagtggaggaggggcgggcgcccagctaccatcaccatcaccaccaccaccaccaccaccaccaccatcaccagcagcagcatcagcagccatccattcctccagcctccagcccggaggacgaggtgctgcccagcacctccatgtacttcaagcagtccccaccgtccacccccaccacgccggccttccccccgcaggcgggggcgttatgggacgaggcactgccctcggcgcccggctgcatcgcacccggcccgctgctggacccgccgatgaaggcggtccccacggtggccggcgcgcgcttcccgctcttccacttcaagccctcgccgccgcatccccccgcgcccagcccggccggcggccaccacctcggctacgacccgacggccgctgccgcgctcagcctgccgctgggagccgcagccgccgcgggcagccaggccgccgcgcttgagggccacccgtacgggctgccgctggccaagagggcggccccgctggccttcccgcctctcggcctcacgccctcccctaccgcgtccagcctgctgggcgagagtcccagcctgccgtcgccgcccagcaggagctcgtcgtctggcgagggcacgtgtgccgtgtgcggggacaacgccgcctgccagcactacggcgtgcgaacctgcgagggctgcaagggctttttcaagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaagttgtccgtacagatagtctgaaagggaggagaggtcgtctgccttccaaaccaaagagcccattacaacaggaaccttctcagccctctccaccttctcctccaatctgcatgatgaatgctcttgtccgagctttaacagactcaacacccagagatcttgattattccagatactgtcccactgaccaggctgctgcaggcacagatgctgagcatgtgcaacaattctacaacctcctgacagcctccattgatgtatccagaagctgggcagaaaggattccgggatttactgatctccccaaagaagatcagacattacttattgaatcagcctttttggagctgtttgtcctcagactttccatcaggtcaaacactgctgaagataagtttgtgttctgcaatggacttgtcctgcatcgacttcagtgccttcgtggatttggggagtggctcgactctattaaagacttttccttaaatttgcagagcctgaaccttgatatccaagccttagcctgcctgtcagcactgagcatgatcacagaaagacatgggttaaaagaaccaaagagagtcgaagagctatgcaacaagatcacaagcagtttaaaagaccaccagagtaagggacaggctctggaacccaacgagtccaaggtcctggttgccctggtagaactgaggaagatctgcaccctgggcctccagcgcatcttctacctgaagctggaagacttggtgtctccaccttccatcattgacaagctcttcctggacaccctacctttctaatcaggagcagtggagcagtgagctgcctcctctcctagcaccctgcttctacgcagcaaagggataggtttggaaacctatcatttcctgtccttccttaagaggaaaagcagctcctgtagaaagcaaagactttcttttttttctggctcttttccttacaacctaaagccagaaaacttgcagagtattgtgttggggttgtgttttatatttaggcattgggggatggggtgggagggggttatagttcatgagggttttctaagaaattgctaacaaagcacttttggacaatgctatcccagcaggaaaaaaaaggataatataactgttttaaaactctttctggggaatccaattatagttgctttgtatttaaaaacaagaacagccaagggttgttcgccagggtaggatgtgtcttaaagattggtcccttgaaaatatgcttcctgtatcaaaggtacgtatgtggtgcaaacaaggcagaaacttccttttaatttccttcttcctttattttaacaaatggtgaaagatggaggattacctacaaatcagacatggcaaaacaataatggctgtttgcttccataaacaagtgcaattttttaaagtgctgtcttactaagtcttgtttattaactctcctttattctatatggaaataaaaaggaggcagtcatgttagcaaatgacacgttaatatccctagcagaggctgtgttcaccttccctgtcgatcccttctgaggtatggcccatccaagacttttaggccattcttgatggaaccagatccctgccctgactgtccagctatcctgaaagtggatcagattataaactggattacatgtaactgttttggttgtgttctatcaaccccaccagagttccctaaacttgcttcagttatagtaactgactggtatattcattcagaagcgccataagtcagttgagtatttgatccctagataagaacatgcaaatcagcaggaactggtcatacagggtaagcaccagggacaataaggatttttatagatataatttaatttttggtaattgggttaaggagaccaattttggagagcaagcaaatcttctttttaaaaaatagtatgaatgtgaatactagaaaagatttaagaaatagtatgagtgtgagtactaggaaggat

SEQ ID NO: 2 - DNA, 4400 bp, RAT
ccgagtctcctgcctcccgccccccacccctccagcgcctgctcctcctccgctccccatacacagacacgctcacacccgctccttcacttgcacacacagacacacgcgcgctcacacgctccgcacacacactccactctctcccgcgcgctcacacccctctctctcggcgccctcgccggtgtcgcgccgcgccgcgccgcagccggacgcccctccagggctcactttgcaacgctgacagagcgggcagtggccgtggaggtgggaaacgtggcgacatcctagcccctggtcgcagccggagactggacgctgcggaacctctcggcggcgctctcccatgagttgggatcgcagcatccccagccagccgctgctcaccgcctctgggagccgctgggtttgtgcaccgcagcccttccgggacagcagctgtgactctcccccaatccagatttcggggtcgctctctagaaactcgctctaaagacggaacctccacagaacccaaagcccactgcgggagagcgcagcccgacaagcccgggcgctgagcctggaccctcaacagagcgggccagcacagcggcggcggctgcttcgcctatcccgacgtccccgcctcctacactctcagcctccgctggagagacccccagccccaccattcagcgcgcaagataccctccagatatgccctgcgtgcaagcccaatatagcccttcgcctccggggtccacttatgccacgcagacttatggctcggaatacaccacagaaatcatgaaccccgactatgccaagctgaccatggacctcggtagcacggggatcatggccacggccacgacgtccctgcccagcttcagtaccttcatggagggctaccccagcagctgcgaactcaagccctcctgcctgtaccaaatgccgccttctgggcctcggcctttgatcaagatggaagagggtcgcgagcatggctaccaccaccaccaccaccatcaccatcatcatcaccaccaccaccagcagcagcagccgtccattcctcctccctctggccccgaggacgaggtactgcccagcacctccatgtacttcaagcagtctccgccgtctacgccgaccactccaggcttccccccgcaggcgggggcgctgtgggacgacgagctgccctctgcgcctggctgcatcgctccgggaccgctgctggacccgcagatgaaggcagtgcccccaatggccgctgctgcgcgcttcccgatcttcttcaagccctcaccgccacaccctcccgcgcccagcccagccggcggccaccacctgggctatgaccccacggccgcagctgcgctcagtctacccctgggagccgcggccgccgcgggcagccaagctgctgcgctcgagggccatccgtacgggctcccgctggccaagaggacggccacgttgaccttccctccgctgggcctcacagcgtcccctaccgcgtccagcctgctgggagagagccccagcctaccatcgccacccaataggagctcatcatccggcgagggcacgtgtgctgtgtgcggggacaatgctgcctgccagcactacggagtccgcacctgcgagggctgcaagggcttcttcaagagaacggtgcagaaaaacgcaaaatatgtttgcttggcaaataaaaactgcccggtagacaagagacgtcgaaatcgatgtcagtactgcaggtttcagaagtgtctcagtgtcgggatggtgaaggaagttgtgcgtacagatagtctgaaagggaggagaggtcgtctgccttccaaaccaaagagcccactacaacaggagccctcgcagccctccccaccatctcctccgatctgtatgatgaacgcccttgtccgagctttaacagacgcaacgcccagagaccttgattactccagatactgtcccaccgaccaggccactgcgggcacagacgctgagcacgtgcagcagttctacaaccttctgacggcctccatcgacgtgtccagaagctgggcagaaaagatccccggattcactgatctccccaaagaagatcagacgttacttatagaatcagcctttttggagctgttcgttcttagactttctatcaggtcaaacactgctgaagataagtttgtgttctgcaatggacttgtcctgcaccgacttcagtgccttcgcggatttggggagtggctcgactccattaaagacttttctttaaatttgcagagcctgaaccttgatatccaagccttagcctgcctgtcagcactgagtatgatcacagagcgacatgggttaaaagaaccaaagagagtggaggagctatgcaacaagatcacaagcagcttaaaggaccaccagaggaagggacaggctctggagccctcagagcccaaggtccttcgcgcactggtggaactgaggaagatctgcacccagggcctccagcgtatcttctacctgaagctggaggacttggtgtccccaccttctgtcatcgacaagctcttccttgataccctgcctttctgagcaggggaagcctgagcagagagctacttgctctgctggcactggtcattaagtgagcaaaaggatgggtttgaacacctgcccctctatccttcctccaggggaaaaagcagctcccatagaaagcaaagacttttttttttcctggcacctttccttacaacctaaagccagaaaccttgcagagtattgtgttggggttgtgttttatatttaggctttggtgggtgggctgggagggggtaaaatagttcatgaggcttttctaagaaattgctgacgaagcacttttggatgatgctatcccagcagtggggtggggagaaaggataatataactgttttaaaaactctttccgggggaatatgactatggttgctttgtatttaaaaataagaacagccaagggctgttttaccagggtagggctgtgtcttaagactgatccctttagtatgtacttcccggatcgaggcacataagtggtgcaaatgaggcggggaaattcttcatttcttcatttctttcttcttcttaaaataaaatggcaaaaaaaaaaagatggaagattatctacaaatcagacttagcaaaatgataatggctattcgcttccacatacaagtgcaattttttagagtgctgtcttactaagtcttgtttgtgaactctccctcattttatatgaaaataagaaggaggcagtcatgttatcaaacggcgtgctcattttcctagctcacccttggtccacctgccctgtagaacccttcggaggtatggcccttctaagactttcaggccactcttgatggaattcgacacccctcccctcaacccatgactatccagatgtcctgaatggggatcaggttataaaatggattgcatatgactgtgttcgctgtgtgtttgtcaacctggacagagttctctaaaccttctttagttgtagcaagttcctgattcctccattcagaagcccaaggagcattgggtgactcgatcaagggttaaccctaggagaacatgcaaataagtaggaactgggtcagacagggtaagcaccagagatgataaggatttatatataaatatatataaaattaatttttgttattggttatagacaattttggaaagcaagagaatcatctcttttttttttttaaagaggaaaagatagtattgatgtattagcaaagattagtggggtacggttcaacattccgtgtttgtgcccccttttctatgtttctactgttgatggcatattattatgaaatgattcgttgcatagtgtccttatttgtatgaacatttgtatgcacgttctattgtaatcgctttgcctgtatttattgcaagaccaccagctcctggaggctgagttacagaataatcaaatggggtgttcgtggtgacttggatacaccggttagaaattaaataagcatatatatatatataaaaacatagcaggttacatatatatttataatgtgtctttttattaaccatttgtacaataaatgtcacttcccacgcagttattttatcctttgtttgcagtgacctttaaggcagcactgtttagcactttgatatgaaattttttgcttatttttttgctaaattcaaataacgtttgaagatttttaggtctaaaagtctttatattatatacactgtatcaagtcaagatacctttggccgttttgctaagactcaaactttgaatgtcaaaccaatgtcacggtagcttctgttagcttttaatcatttttgctttagtctttttttttaaaaaaaa

SEQ ID NO: 3 - DNA, 349 bp, Hepatitis A virus
caatcactctgatgagtatttgtcatttagttgttatttatctgtcacagaacaatcagagttttattttcccagagctccattgaattcaaatgctatgttatccactgaatcaatgatgagtagaattgcagctggagatttggagtcatcagtggatgatcctagatcagaggaggataaaagatttgagagtcacatagaatgcaggaagccatataaagaactgagattagaagtagggaaacaaagactcaagtatgctcaggaagaattgtcaaatgaagtacttccaccccctaggaaaatgaaggggctgttttcacaagccaaaatttcacttttttat

SEQ ID NO: 4 - DNA, 433 bp, Homo sapiens
taacaatcttgttgcatagctcttcgactctctttggttcttttaacccatgtctttctgtgatcatgctcagtgctgacaggcaggctaaggcttggatatcaaggttcaggctctgcaaatttaaggaaaagtctttaatagagtcgagccactccccaaatccacgaaggcactgaagtcgatgcaggacaagtccattgcagaacacaaacttatcttcagcagtgtttgacctgatggaaagtctgaggacaaacagctccaaaaaggctgattcaataagtaatgtctgatcttctttggggagattagtaaatcccggaatcttttctgcccagcttctggatacatcaatggaggctgtcaggaggttgtagaattgttgcacatgctcagcatctgtgcctgcagcagcctggtcagtgggaca

SEQ ID NO: 5 - DNA, 414 bp, Suaeda maritima
  modified_base (127)..(413): N = A, C, G and/or T/U
ctcatccttcactctttctaattttctctctttcatttcctcacatacattttattcccattttgtcccttgctctttcattatcaaaacatacaaacttaattttattattataaattagcctctnanncncctccanttntttttncanacttgagnnnaattccgttttatacagcggntgaagaaaaagaaaaagaaaataatgacaaagcaagaaatggtggtttcagaagaaggaaaagcgaattcaagcaaatcatcatcatcaacatcatcatgtacatntnaaaaaccaccaccagngccaagcaaatacatatnagnaccaatgttngaatgngagaggcaactgnttaaagacctggatatnaatggnggntnttgggttgattntatgagagcttnttntnc

SEQ ID NO: 6 - DNA, 1606 bp, Sus scrofa
ataccctccagatatgccctgcgtgcaagcccagtatagcccttcgcctccaggttccagttatgcagcccagacatatggctcggaatacaccacagagatcatgaatcctgactacaccaagctgaccatggaccttggcagcaccgagatcacggccactgctacaacgtccctgcccagcttcagtaccttcatggagggctactccagcaactacgaactcaagccttcctgcctgtaccaaatgcagccgtcggggcctcggcccctgatcaagatggaggagggccgcgcgcacggctaccaccatcaccaccacgaccaccaccaccatcaccaccaccagcagcagcagcagcagcagcagccacctccgcagcagcagcagccatccattccgcccccctccggtccggaggacgaggtgctgcccagcacctctatgtacttcaagcagtccccgccgtccacccccaccacgccggtcttcccccagcaggcgggggcgctgtgggaagacgcgctgccctctgcgcagggctgcatcgcgcccggcccgctgctcgacccgccgatgaaggcggtgcccacggtggccggcgcgcgcttccctctcttccacttcaagccctcgccgccgcatccgcccgcgcccagccccgccggcggccaccatctcggctacgacccgacggccgctgccgcgcttggcctgccgctaggagccgccgccgccgccgccgccgccgccgcaggcagtcaggccgccgcgctcgagggccacccgtacgggctgccactggccaaaagggcggccgcgctggccttctcgccgctgggcctcacgacctcccccaccacgtccagcctactgggcgagagccccagcctgccgtctccgcccaacaggagcaccgcgtcaggcgaggggacgtgcgccgtgtgcggggacaacgccgcctgtcagcactatggcgtgcgcacctgcgagggctgcaagggcttcttcaagagaacggtgcagaaaaatgcaaaatatgtttgcctggcaaataaaaactgccccgtagacaagaggcgtcgaaaccgatgtcagtactgtcgatttcagaagtgtctcagtgtcggaatggttaaagaagttgtccgtacagatagtctgaaagggaggagaggtcggctgccttccaaaccaaagagcccgttacagcaggaaccctctcagccctctccaccttctcctccggtctgtatgatgaatgcccttgtccgagctttaacagactcaacgcccagagatcttgattattccagaggccacgcctgcggcatatggaagttcccaggctaggagtcgaatcagagatgcagctgccagcctacgccacagacacagccacgtgggatctgagccgtgtctgcagtccacaccacagcttgtggcaaaaccagatccttaacctatggagtgaggccaagggatcgaaccttcatcctcacagatactagacggcttcataacccattgagccacaggaggagctccacccagagatcttaataaaacagtttcttgggcctaaaaaaaaaaaaaaaaaaaaaa

SEQ ID NO: 7 - DNA, 1967 bp, Homo sapiens
tggcgccggccggggtcccggccaaccgccgaatttagtaacatcgcctgcgtcaatcacgcgcctcggtgcgtcaggccgcgcggctccaggtcctgctcccccccttcaagcctttgaatggatacaatgtagcagcgccctccttccttccgaggctggattggaaccgccgcagtgcagagactcggttgctctcggctgggtcaactttcggggcattctcccacgatcctctccgcaccaccgtgtctgaattggaagtggaggcgaagaaagatatacatgccatatttacctatatgtagtttgttttcaagtttctggtcctagctcgaaccttcttcgattctgaaatgtgtgctgtctacaaaggaatcttgtatctcccctcggcgcagccccccgccccgccacacacacacaaattgggacaggtcaaacatataaaacggtatttgtgattcaagcggaccacatggggaccactctatctgcattgtttcactcaaatattttctcctgtccaaaaattcatttctgaaagagactgcgttcactcagcagcaacctttgggactaggggtctttaactctgataaattttgttttcatcaagaaatttacacttaaatttatcatttccaggaagaaattgctctccttcatacagtcacccaggctttcggcacaccatttcatgacaaatgtgtccgaggagaccaaagcaaatcccctagcgagggactgactaataagtcctgttgattgatttcgaaatgtttaatttgggagatgtgggcggagggcatctacaaccatcaaaaagtgaaagtgctagttgagagttccatttctgacccggtgccggggaggaggaatgatttgcaatagtcagacccgctcagctgttcaacacgtgtgtgtttgttttacacacagagtagtttctgctgcagccgcgtgtgcatgatggatgtgcacttcgctgggttataacgtgtccagttaagaaacccacgccgtacgtgtaaagaaatcaaaccttatccccggaaccatctgcatccctgtgtgaacacgcacccagtaaatgatgcggggaggggggattagcctgggcgcagaggaccggagcaacgtaaacagctttagaacctatgcaagaggaaagtgcagctgcacctcagggcgtcttcgggctggtgccagacgccttctgcaccggctgccaggtcactggagctggtcagaagctggctggcggagcttccctttcggaagagctgtcctctcccttacccccctcgccctggctccgtgcctcggggcagcctcggaggcgcgccagcagcactcctccaactctactccacccgagcctgacagctgggcggtcccgcctgacccgtgggcaggccgctgcaccctcccgcagacgcacgccctggcgagcggttccgctgcaaaaagagaagcccccaggccggggccggccgtgcggcggagtttccattgtgcggccgtgcgactggccgaggaacgcgcgcgcgcgcgcacacgaacacacacaccctccctcgcacacgcggaaccggctgggccaggggagggaggaggagggtgacgtagcgtcccatggcgtcacattgacgtctcgcattccaggcactctatggagaggccgctagggctcctgtggcataaatgacgtgccgagagagcgagcgaacgcgcagccgggagagcggagtctcctgcctcccgccccccacccctccagctcctgctcctcctccgctccccatacacagacgcgctcacacccgctccctcactcgaacacacagacacaagcgcgcacacaggctccgcacacacacacttcgctctcccgcgcgctcacacccctcttgccctgagcccttgccggtgcagcgcggcgccgcagctggacgcccctcccg

SEQ ID NO: 8 - DNA, 5115 bp, Sus scrofa
ggcagcgccgccgccgcttcgcctcgcaggacgtccccgcctcctccactctcagcctctgctggagagacccccagccccaccattcagcgcgcaagataccctccagatatgccctgcgtgcaagcccagtatagcccttcgcctccaggttccagttatgcagcccagacatatggctcggaatacaccacagagatcatgaatcctgactacaccaagctgaccatggaccttggcagcaccgagatcacggccactgctacaacgtccctgcccagcttcagtaccttcatggagggctactccagcaactacgaactcaagccttcctgcctgtaccaaatgcagccgtcggggcctcggcccctgatcaagatggaggagggccgcgcgcacggctaccaccatcaccaccacgaccaccaccaccatcaccaccaccagcagcagcagcagcagcagcagccacctccgcagcagcagcagccatccattccgcccccctccggtccggaggacgaggtgctgcccagcacctctatgtacttcaagcagtccccgccgtccacccccaccacgccggtcttcccccagcaggcgggggcgctgtgggaagacgcgctgccctctgcgcagggctgcatcgcgcccggcccgctgctcgacccgccgatgaaggcggtgcccacggtggccggcgcgcgcttccctctcttccacttcaagccctcgccgccgcatccgcccgcgcccagccccgccggcggccaccatctcggatacgacccgacggccgctgccgcgcttggcctgccgctaggagccgccgcsgccgccgccgccgccgccgcaggcagtcaggccgccgcgctcgagggccacccgtacgggctgccactggccaaaagggcggccgcgctggccttctcgccgctgggcctcacgacctcccccaccacgtccagcctactgggcgagagccccagcctgccgtctccgcccaacaggagcaccgcgtcaggcgaggggacgtgcgccgtgtgcggggacaacgccgcctgtcagcactatggcgtgcgcacctgcgagggctgcaagggcttcttcaagagaacggtgcagaaaaatgcaaaatatgtttgcctggcaaataaaaactgccccgtagacaagaggcgtcgaaaccgatgtcagtactgtcgatttcagaagtgtctcagtgtcggaatggttaaagaagttgtccgtacagatagtctgaaagggaggagaggtcggctgccttccaaaccaaagagcccgttacagcaggaaccctctcagccctctccaccttctcctccggtctgtatgatgaatgcccttgtccgagctttaacagactcaacgcccagagatcttgattattccagatactgccccgctgaccaggccgctgcaggcacagatgctgagcatgtacaacagttctacaaccttctcacagcctccattgacgtatccagaagctgggcagaaaagattcctggatttactgatctccctaaagaagatcagacattacttatagaatcagcctttttggagctgtttgttctcagactttccatcaggtcaaacactgctgaagataagtttgtgttctgcaatggacttgtcctgcatcgacttcagtgccttcgtggatttggggagtggctcgactccattaaagacttttccttacgtttgcagagcctgaaccttgatatccaagccttagcctgcctgtcagcactgagcatgatcacagaacgacatgggttaaaagaaccaaagagagtggaggagctatgcaacaagatcacaagcagcttaaaagagcaccagagtaagggacaggctttggagcccaccgagcccaaggtcctgcgtgccctggtggaactgcggaagatctgcaccctgggcctccagcgcatcttctacctaaagctggaagacttggtgtctccaccttccatcatcgacaagctcttcctagataccctgcctttctgagcaggagcagcctgagcagggagctgcttcatctgctagcagccacttgctaagcggcaaaggaatgggtctggacatctaccattttctgtccttccttaagagaaaaagcagctcctgtagaaataaaagacttcttcttcttcttcttttttttttttttttttttctggcacttttccttatgactgaaagccagaaaacttacagagtattgtgttggggttgtgttttatatttaggctttggggttggggtgggaggggggtatagttcatgagggttttctaagaaattgctaacaaagcacttttggacgatgctatcccagcaggaaaaaaaaaaaaaaaaaggataatgtaactgttttaaaactctttctggggaatacagttatagttgatttgtatttaaaaacaagaacagccaagggttgtttgccagggtagggtgtgtttgagattgatccctttagaatatacttcttgtatcaagggtacatatgtggtgcaaaaaaagcagaaattccctcttcctaatttccttcttcatttattttaacaaatggtaaaagaaggaggattacctataaatcggacatagcaaaatgataatggcttttcgcttccatatacaagtgcaatttttaaagtgctgtcttactaagtcttgtttattaactctcctttattttatatggaaataaaggaggcagtcatgatagcaaatgacacatgctaattttcctagcagaggcgttgtccacctgacccatagaactcttctgagacgtggtccatccaagatttttggccgttcttgatggatccgggtccctgccctgagctgctttgaaaggggctcagattatatgaggggttacgtacagctttttttgtcgctttctataaatccagccagatttccctaaacttgcttcaattatggtaacagactgacacattcattcagaagccccaagagcattcagtgaatttcaagtgtttgaccccaagataagaacatgcaaataagtaagaatggatcatacagggtaagcaccagggataataagttttttaaatatatataatttaatttttattatcagttaaagagacaattttggagagcaagtgattcttattaaaaaattagtgtgaatgtgagtactagaaaggattagtgggctgcgtttcaacattccgtgttcgtactcccttttgtatgtttatactgttaatgccatattattatgagataatttgttgcatagtgtcctttatttgtataaacatttgtatgcacgttatattgtaatagctttgcctgtatttattgcaagaccaccagctcctggaagctgagttacagagtacttaaatggggtgttcacagtgaatttggatacaccaattagaaattaaataagcaaatatatatataaatatagcaagttacatatatatttataatgtgtctttttattaaccatttgtacaataaatgttacttcccatgcagttattttacggttcatttgcagtgacttttaaggcagtactgtttagcactttgatattaaaattttgcttacgttttgctaaattcaaatgatgtttgaagatttttaggtctaaaagtctttatattatatactctgtatcaagtcaaaatatctttggtcattttgctaagaaacaaactttgaatgtcaaactgatgtcatagtagtttttgttagctctgaatcatttttgctttagtctttttaaaggaaaaataacaaaactatgctgtttatattgtcattaaattatacaatcaaacaaatgccaaatgaattgcctaattgctgcaatgaataacccagatagcaaaatcatgtatgtttttttcccaaaattcattctgatatttgatcattatgtttgtgtgagcttttatgaaagattattatttttatatcaagatgataggatctggaatgttaggatctcagaatgttagatttaaaaggggcctgccttgtcaactagtccacccccacacactaaaattcatagaggaggaaattggggcccagcaaagggcaaagggttaaccaaggacaaagagctggtaacagaatcaagaccaggacctaattctccttgccacagtctttttactcactttactgcatctgtaggaaaacaggcttttaaaaataaccagtaatatgtacattttaccgtgagtaaagcagtaactttgcagtaatttttgagcttatatgcaaacataatgaacattattaaatatcaggagagctaacatttcatacgagttagcttcagaccaaattcaaattgaatttgaataaattagaaatactgtgcatacataacccttttgtgcactgttaagttttgaaatcttaatcggtttttgttttttgtttttgtcatgtctgtaaaggaaaaacaaaagaaaaaaacagagccctggagaaatgctgtcactttttatttttacacccatcagatttaaggaaaagactttttagctgatattgattggttggaaggaatgaagaaggtttttacttataggtccaggcactagtgctgaaaataaagattatagccagtgttcttctgtcttccatagttattacaactatgagagcccccccaagtcatctatcaattcaactctcttttttctttttgtcttaatgttgacacacaagtttatacagagtggatgaccagactagctcagaagaggacagcaagaattaaagcaggtgattcttcccttgtgggagagctctctcagtgtgaacatgccttctttgggcagaaatcaggaatccaccagctgttaatggagagtgccttgcttttatttcagacagcagagttttccaaagtttctctgctcctctaacagcgttgctctttagtgtgtgttaacctgtgtttgaaagaaatgctcttgtacattaacaatgtaaatttaaatgattaaattaaattacattttatcaatggctaaaaaaaaaaaaaaaaaaaaaaaaaaaaaaaa

SEQ ID NO: 9 - DNA, 1453 bp, Homo sapiens
gtaggtccgaaggcaagacccttttctcctccctggctgagggaagtgggtgggggaaccacacactcggcgggcagcgtggtcgacctgcccagtgccaggacagtgactgctggccgcgaatttcacaacacaggtggcttcctcacaggaagctcctctgtataccacaccctgttgctactgagtggagcagccaaattaaattaagcttgcattgctcaaaattaattttcctaagagaaatacaaatacaccaatagattagggtattttatacatttttaatttcatttttgctcttcttttatagccagtgtgcacatgtaagagttatggaatcacttagagcaatactgagcattttcatttatataaaacccaaatcatttgggtgcagaagtttggatgattgaagctcagagggaaggagaaaagcatttgagatgaaaccaaagagtaaatttgagtttggcaaagaacagattgcacttttggtttgtaccacctctttacaaatttgttaaaggatactaggtgccaagcctgtgtgggcaccatagataatgcagataaatagaatacaatcccagcaagctcatcttctagctagaaacctcagaaagacacaaataagagcagtaaaaaggggataacgcaggaaagaacatgtgagcaaaaggcctggagaaatcctagaggtgctgaagggagaaggagaatagatcaaagatcaggcagtgttctttcttttaaatgattagcctttcatttcatcccaacaactggacagcaagataagtaactggacttcaaactagtgagtgtatttttaaggccctgcttgttaaagaaaggcttgaactggcctctcctcatcactgcttcttccaacaggccctcatcaccttttttcaagtcaagatttcatcccatacatgcatgactcaatcagatttggaaatgtgggtaagagaaagatgtcaaaggaaatgtgaagtattcacttctctattagtcacaccttttacaccatagactccaaagaggcgttaagcacctggttttcctttggctcagaaaaaccaaccaccaaaaaccgccgttttttaccatttatatttagccataaagaaagaaaaataattagataaatcatccactacatccaataattctcagcgccttctcactcagttcagcctctctgaacaatagtaagcaccctggataccagccactttgggggcaacatagtcaaactggcagagaaatcaagtctattgagaaactgctttttttcatgggtactaattctagtgtcataaggaaatacctatactaacttgcctattatgatagttataaactgttatcacaaaacagtcactgatatgttttattagttagaattgggatatatatatgtgtgtgtgtgtgtgtgtgtgtgtgt

SEQ ID NO: 10 - DNA, 1703 bp, Homo sapiens
tggcgccggccggggtcccggccaaccgccgaatttagtaacatcgcctgcgtcaatcacgcgcctcggtgcgtcaggccgcgcggctccaggtcctgctcccccccttcaagcctttgaatggatacaatgtagcagcgccctccttccttccgaggctggattggaaccgccgcagtgcagagactcggttgctctcggctgggtcaactttcggggcattctcccacgatcctctccgcaccaccgtgtctgaattggaagtggaggcgaagaaagatatacatgccatatttacctatatgtagtttgttttcaagtttctggtcctagctcgaaccttcttcgattctgaaatgtgtgctgtctacaaaggaatcttgtatctcccctcggcgcagccccccgccccgccacacacacacaaattgggacaggtcaaacatataaaacggtatttgtgattcaagcggaccacatggggaccactctatctgcattgtttcactcaaatattttctcctgtccaaaaattcatttctgaaagagactgcgttcactcagcagcaacctttgggactaggggtctttaactctgataaattttgttttcatcaagaaatttacacttaaatttatcatttccaggaagaaattgctctccttcatacagtcacccaggctttcggcacaccatttcatgacaaatgtgtccgaggagaccaaagcaaatcccctagcgagggactgactaataagtcctgttgattgatttcgaaatgtttaatttgggagatgtgggcggagggcatctacaaccatcaaaaagtgaaagtgctagttgagagttccatttctgacccggtgccggggaggaggaatgatttgcaatagtcagacccgctcagctgttcaacacgtgtgtgtttgttttacacacagagtagtttctgctgcagccgcgtgtgcatgatggatgtgcacttcgctgggttataacgtgtccagttaagaaacccacgccgtacgtgtaaagaaatcaaaccttatccccggaaccatctgcatccctgtgtgaacacgcacccagtaaatgatgcggggaggggggattagcctgggcgcagaggaccggagcaacgtaaacagctttagaacctatgcaagaggaaagtgcagctgcacctcagggcgtcttcgggctggtgccagacgccttctgcaccggctgccaggtcactggagctggtcagaagctggctggcggagcttccctttcggaagagctgtcctctcccttacccccctcgccctggctccgtgcctcggggcagcctcggaggcgcgccagcagcactcctccaactctactccacccgagcctgacagctgggcggtcccgcctgacccgtgggcaggccgctgcaccctcccgcagacgcacgccctggcgagcggttccgctgcaaaaagagaagcccccaggccggggccggccgtgcggcggagtttccattgtgcggccgtgcgactggccgaggaacgcgcgcgcgcgcgcacacgaacacacacaccctccctcgcacacgcggaaccggctgggccaggggagggaggaggagggtgacgtagcgtcccatggcgtcacattgacgtctcgcattccaggcactctatggagaggccgctagggctcctgtggc

SEQ ID NO: 11 - DNA, 785 bp, Homo sapiens
tgccagcactacggcgtgcgaacctgcgagggctgcaagggctttttcaagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaagttgtccgtacagatagtctgaaagggaggagaggtcgtctgccttccaaaccaaagagcccattacaacaggaaccttctcagccctctccaccttctcctccaatctgcatgatgaatgctcttgtccgagctttaacagactcaacacccagagatcttgattattccagagtaagttttatgatttcctgctttcaaatgaatgatcagggtctctatttatggctactagtaataagagttgattgaatgattttgtgtctggcaccatgttagacagttttcatactttttctatatttctcgcttcatttagcaattcagtgcatccattgcagcaaataatttttgccttattgaatctctaaatgccttaacaagtgaccctgacagtgctgcacctgtcatacacattgttgcaggattcctggtggttgtgccaatgaaaatctgcacagacaaactacaatttgtagatttatctcgtgatctagacaaagtgactactgttttttttcatattgtgttcaaaccatctgggtgagcctcaagttattactaagcagtttatccaattgcatcagcattgattgacctgctgc

SEQ ID NO: 12 - DNA, 966 bp, Homo sapiens
gtgtgtatatatatatatatgggtgggtgttttgttgcagctgctgatctttttctttgcagatggtacaaactctcccgagtcaatttcctgggcctatgtccccacctagctgactgaagttatcaacaggggtccagtttgtgcaggctgctagccctattggaagagtggggacgaggtgggagaaagcaaccacaacgtgtgtgggcaacctcaattggcactcataaaatgttagaatgtcaactctctcccttggccactaaatctctcacagggtagtttttcttgcctaactcaggtttacaaatcaatgtgtatgccttgggggaccaatggcctctttcctcccaaataaaccactggctttctctttgtccccctaggttatagctgaggagcccactccaattagtttataggattcaaagcctctttttaaaaacatctctgagcttatgaggaaagacttcaagtttcccaaatctagtggaggacagggcaagggaggaaagataggtacaggagtccacaggaggccaggttttggcacccctttgtcaggaattcagcttccttactagggatgaagaaaataagtgtggggctttgtgtctatgctaccagaaggaggagaggatgacacttcctctctgtttcccagattagagaacagtgaacccaatgctgcctgttggctagaaaacaagtgttaacttgcttctgaaagacccttctctctgtccctgcagatatgccctgcgtccaagcccaatatagcccttcccctccaggttccagttatgcggcgcagacatacagctcggaatacaccacggagatcatgaaccccgactacaccaagctgaccatggaccttggcagcactgagatcacggctacagccaccacgtccctgcccagcatcagtaccttcgtggagggctactcgagcaactacga

SEQ ID NO: 13 - protein, 625 aa, Homo sapiens
MPCVQAQYSPSPPGSSYAAQTYSSEYTTEIMNPDYTKLTMDLGSTEITATATTSLPSISTFVEGYSSNYELKPSCVYQMQRPLIKVEEGRAPSYHHHHHHHHHHHHHHQQQHQQPSIPPASSPEDEVLPSTSMYFKQSPPSTPTTPAFPPQAGALWDEALPSAPGCIAPGPLLDPPMKAVPTVAGARFPLFHFKPSPPHPPAPSPAGGHHLGYDPTAAAALSLPLGAAAAAGSQAAALEGHPYGLPLAKRAAPLAFPPLGLTPSPTASSLLGESPSLPSPPSRSSSSGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDSTPRDLDYSRYCPTDQAAAGTDAEHVQQFYNLLTASIDVSRSWAERIPGFTDLPKEDQTLLIESAFLELVLRLSIRSNTAEDKFVFCNGLVLHRLQCLRGFGEWLDSIKDFSLNLQSLNLDIQALACLSALSMITERHGLKEPKRVEELCNKITSSLKDHQSKGQALEPNESKVLVALVELRKICTLGLQRIFYLKLEDLVSPPSIIDKLFLDTLPF

SEQ ID NO: 14 - protein, 626 aa, Homo sapiens [sequence omitted]

SEQ ID NO: 15 - protein, 628 aa, RAT
MPCVQAQYSPSPPGSTYATQTYGSEYTTEIMNPDYAKLTMDLGSTGIMATATTSLPSFSTFMEGYPSSCELKPSCLYQMPPSGPRPLIKMEEGREHGYHHHHHHHHHHHHHHQQQQPSIPPPSGPEDEVLPSTSMYFKQSPPSTPTTPGFPPQAGALWDDELPSAPGCIAPGPLLDPQMKAVPPMAAAARFPIFFKPSPPHPPAPSPAGGHHLGYDPTAAAALSLPLGAAAAAGSQAAALEGHPYGLPLAKRTATLTFPPLGLTASPTASSLLGESPSLPSPPNRSSSSGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDATPRDLDYSRYCPTDQATAGTDAEHVQQFYNLLTASIDVSRSWAEKIPGFTDLPKEDQTLLIESAFLELFVLRLSIRSNTAEDKFVFCNGLVLHRLQCLRGFGEWLDSIKDFSLNLQSLNLDIQALACLSALSMITERHGLKEPKRVEELCNKITSSLKDHQRKGQALEPSEPKVLRALVELRKICTQGLQRIFYLKLEDLVSPPSVIDKLFLDTLPF

SEQ ID NO: 16 - protein, 446 aa, Sus scrofa
MPCVQAQYSPSPPGSSYAAQTYGSEYTTEIMNPDYTKLTMDLGSTEITATATTSLPSFSTFMEGYSSNYELKPSCLYQMQPSGPRPLIKMEEGRAHGYHHHHHDHHHHHHHQQQQQQQQPPPQQQQPSIPPPSGPEDEVLPSTSMYFKQSPPSTPTTPVFPQQAGALWEDALPSAQGCIAPGPLLDPPMKAVPTVAGARFPLFHFKPSPPHPPAPSPAGGHHLGYDPTAAAALGLPLGAAAAAAAAAAGSQAAALEGHPYGLPLAKRAAALAFSPLGLTTSPTTSSLLGESPSLPSPPNRSTASGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPVCMMNALVRALTDSTPRDLDYSRGHACGIWKFPG

SEQ ID NO: 17 - protein, 643 aa, Sus scrofa
MPCVQAQYSPSPPGSSYAAQTYGSEYTTEIMNPDYTKLTMDLGSTEITATATTSLPSFSTFMEGYSSNYELKPSCLYQMQPSGPRPLIKMEEGRAHGYHHHHHDHHHHHHHQQQQQQQQPPPQQQQPSIPPPSGPEDEVLPSTSMYFKQSPPSTPTTPVFPQQAGALWEDALPSAQGCIAPGPLLDPPMKAVPTVAGARFPLFHFKPSPPHPPAPSPAGGHHLGYDPTAAAALGLPLGAAAAAAAAAAGSQAAALEGHPYGLPLAKRAAALAFSPLGLTTSPTTSSLLGESPSLPSPPNRSTASGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPVCMMNALVRALTDSTPRDLDYSRYCPADQAAAGTDAEHVQQFYNLLTASIDVSRSWAEKIPGFTDLPKEDQTLLIESAFLELFVLRLSIRSNTAEDKFVFCNGLVLHRLQCLRGFGEWLDSIKDFSLRLQSLNLDIQALACLSALSMITERHGLKEPKRVEELCNKITSSLKEHQSKGQALEPTEPKVLRALVELRKICTLGLQRIFYLKLEDLVSPPSIIDKLFLDTLPF

SEQ ID NO: 18 - protein, 143 aa, Homo sapiens
CQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDSTPRDLDYSRVSFMISCFQMNDQGLYLWLLVIRVD

SEQ ID NO: 19 - protein, 69 aa, Homo sapiens
MPCVQAQYSPSPPGSSYAAQTYSSEYTTEIMNPDYTKLTMDLGSTEITATATTSLPSISTFVEGYSSNY

SEQ ID NO: 20 - DNA, 1831 bp, Homo sapiens
gaattccgggctcgctcagctgctgcccagcctcggctgtgaggataggctggctgggcagcacgtctctccccacagggctccctgagaccaccaggaagagcccccaaccaatcttgggattctcccttcgtgcggttgtctgggacctttttccagggtcaaagcagatcgtgaggaggaagctgagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaaggtaccgcgcagccccaggtggggccttttgttggaaatggagagaggctggcctcatcccattgggacctgtggtctccccctgggttctcctcctagctaagtcctgtcctgcagggtgggatcagccctgccaggtgggccgccttcctggagacccgtagatgccagggctggaagctttcatttgccgggacactcgggcccatgggattgcacagagctggagggaggggtgagataggggcagataggagctgcaggggtgcctggcgagcctctggttttcctctgctcctctgcctgtcctctcccaactcaaggttctagtgggaagggtgcccccaggctctcatgttcctggcgtgagatgaaaggatcctgcggagggtttggttcttgagggctgggggtggacttgggaacaggctgtgtgtttgtcccagcgatggtgcctgcttagcttcccgtccccaccccccagccccttggccctctcctgtctgccctagggagaaggcaggtggacaagggcccatgaaaaaatacaggtgtctagactgccagggagaccctggccccccagtagtgtgtcctggggacttcctcagagcgagaaacctccccccaatgtcttcaagacttttctctcccccccccccaaccccgtctctccctcccttgccacccaaatgttagaaaaatagctgtgaacagagagcgcttttgtctgcaatggcagcaggatctggacggtccggaattc

SEQ ID NO: 21 - DNA, 250 bp, Homo sapiens
acacgggctcaaggaacccaagagagtggaagaactgcaaaacaagattgttaattgtctcaaagaccacgtgactttcatcaatggggggttgaaccgccccaattatttgtgcaaactgttggggaagctcccataacttcgtaccctttgcacacatgggctgcagcgcattttctacctgaaattggaagacttggtgccaccgccatcgatatttgacaaacttttcctgtacactttacctttc

SEQ ID NO: 22 - DNA, 442 bp, Zebra Fish
ggaacttcaaaacaagctcataaactgtctgaaggatcaggtgtcctgcagtggtgaattgtctaaactgttggagaagctgccggaggtgcgcgcgctgtgcacgcagggtctgcagcgcatcttttacttgaaactggaggatttggtgcccacgcctgcgatcattgataaactctttcatgacactttaccattctaaacaatctcgtctgaactgaagcaggttctcgaaaccttgttattcttcgatgttgcggactttttaggaaatgaaaatggcggatggtatttttaaagattggagaaagactgcccgggacaaaactgaacagtcctatttggaggaaataattattcaagaaaaaaatattatggaggaaaagggactattcttatgcctgtttaaagtgtattcagttatatactgaaaataaatgcg

SEQ ID NO: 23 - DNA, 492 bp, Mus musculus
acaaaattgtaaattgtcttaaagaccatgtgactttcaataatgggggtttgaaccgacccaactacctgtctaaactgttggggaagctgccagaactccgcaccctttgcacacagggcctccagcgcattttctacctgaaattggaagacttggtaccaccaccagcaataattgacaaacttttcctggacaccttacctttctaagaccttctcccaagcacgtcaaagaactggaaagaaaaaaaaaataacatccagagggggctggtcacatgggcagagagctggttgaagtgtccagttcaccttatctcccttctgtagacccctagccctcaccccttaagtaaacaaacaaacaaacaaaccacaaataaaaactgtagctatttcctaacctgcaggcagaacctgaaagggcattttggctccggggcatcctggatttagaaaacggacagcacacagtacagtggtataaact

SEQ ID NO: 24 - DNA, 500 bp, Homo sapiens
  modified_base (420): N = A, C, G and/or T/U
agaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaagacctcctcccaagcacttcaaaggaactggaatgataatggaaactgtcaagagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcangcagaacctgaaagggcattttggctccggggcatcctggatttagaacatggactacacacaatacagtggtataaact

SEQ ID NO: 25 - DNA, 499 bp, Homo sapiens
  modified_base (420): N = A, C, G and/or T/U
agaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaagacctcctcccaagcacttcaaaggaactggaatgataatggaaactgtcaagagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcangcagaacctgaaagggcattttggctccggggcatcctggatttagaacatggactacacacaatacagtggtataaac

SEQ ID NO: 26 - DNA, 520 bp, Mus musculus
acaaaattgtaaattgtcttaaagaccatgtgactttcaataatgggggtttgaaccgacccaactacctgtctaaactgttggggaagctgccagaactccgcaccctttgcacacagggcctccagcgcattttctacctgaaattggaagacttggtaccaccaccagcaataattgacaaacttttcctggacaccttacctttctaagaccttctcccaagcacgtcaaagaactggaaagaaaaaaaaaataacatccagagggggctggtcacatgggcagagagctggttgaagtgtccagttcaccttatctcccttctgtagacccctagccctcaccccttaagtaaacaaacaaacaaacaaaccacaaataaaaactgtagctatttcctaacctgcaggcagaacctgaaagggcattttggctccggggcatcctggatttagaaaacggacagcacacagtacagtggtataaactttttattatcagttcaaaatcagtttgt

SEQ ID NO: 27 - DNA, 396 bp, Homo sapiens
tgaatactaacatgtcaattgttttgtggagataagagtgaacgtttcccagggctggatggcactgtatttagtctgtatggaaatggcaatttacatatttaaagcagcgacctcgtagcaccatccctaattgaattaattgccccggaacatctaatttccttactggtcagagagaggtttaattgttataaaaacctggctcccctattagaaacggggttagcaatttcacgggttatatattttagagaacctcattaagtgctttttaaaatgaaattccagttccaggcgaaccctgactatcaaatgagtggagatgacacccagcatatccagcaattctatgatctcctgactggctccatggagatcatccggggctgggca

SEQ ID NO: 28 - DNA, 2481 bp, Homo sapiens
cgaacttggggggagtgcacagaagaacttcgggagcgcacgcgggaccagggaccaggctgagactcggggcgccagtccgggcaggggcagcgggagccggccggagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcttccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaagcgcacagtgcagaaaaacgccaagtacatctgcctggctaacaaggactgccctgtggacaagaggcggcgaaaccgctgccagttctgccgcttccagaagtgcctggcggtgggcatggtgaaggaagttgtccgaacagacagcctgaaggggcggcggggccggctaccttcaaaacccaagcagcccccagatgcctcccctgccaatctcctcacttccctggtccgtgcacacctggactcagggcccagcactgccaaactggactactccaagttccaggagctggtgctgccccactttgggaaggaagatgctggggatgtacagcagttctacgacctgctctccggttctctggaggtcatccgcaagtgggcggagaagatccctggctttgctgagctgtcaccggctgaccaggacctgttgctggagtcggccttcctggagctcttcatcctccgcctggcgtacaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccgaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgcctgggaacacgtgtgcacatgcgcactctctcatatgccaccccatgtgcctttagtccacggaccccagagcacccccaagcctgggcttagctgcagaacagagggacctgctcacctgcccaaaggggatgaagggagggaggctcaaggcccttgggggagggggatgccttcatgggggtgacccacgatgtgttcttatcccccccgcctggccaccggcctttatgttttttgtaagataaaccgtttttaacacatagcgccgtgctgtaaataagcccagtactgctgtaaatacaggaagaaagagcttgaggtgggagcgggctgggaggaagggatgggccccggccttcctgggcagcctttccagcctcctgctgggctctctcttcctaccctccttccacatgtacatgtacataaactgtcactctaggaagaagacaaatgacagattctgaccatttatatttgtgtattttccaggatttatagtatgtgacttttctgattaatatatttaatatattgaataaaaaatagacatgtagttgg

SEQ ID NO: 29 - DNA, 2469 bp, Homo sapiens
ttcggcacgagtgcacttcggcagagttgaatgaatgaagagagacgcggagaactcctaaggaggagattggacaggctggactccccattgcttttctaaaaatcttggaaactttgtccttcattgaattacgacactgtccacctttaatttcctcgaaaacgcctgtaactcggctgaagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctctgcgctgtgtgtggggacaacgcggcctgccaacactacggcgtgcgcacctgtgagggctgcaaaggcttctttaagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccaggttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacaggtccaacccagtggagggtaaactcatcttttggaatgggtgggtcttgcacaggttgcaatgcgttcgtggctttggggaatggattgattccattgttgaattctcctccaacttgcagaatatgaacatcgacatttctgccttctcctgcattgctgccctggctatggtcacagagagacacgggctcaaggaacccaagagagtggaagaactccaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaagacctcctcccaagcacttcaaaggaactggaatgataatggaaactgtcaagagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcaggcagaacctgaaagggcattttggctccggggcatcctggatttagaacatggactacacacaatacagtggtataaactttttattctcagtttaaaaatcagtttgttgttcagaagaaagattgctataatgtataatgggaaatgtttgcccatgcttggttgttgcagttcagacaaatgtgacacacacacacatacacacacacacacacacacagagacacatcttaaggggacccacaagtattgccctttaacaagacttcaaagttttctgctgtaaagaaagctgtaatatatagtaaaactaaatgttgcgtgggtggcatgagttgaagaaggccaaggcttgtaaatttacccaatgcagtttggctttttaaattattttgtgcctatttatgaataaatattaccaattctaaaagataagtgtgtttcccacaaaaaaaaaaaaaaaa

SEQ ID NO: 30 - protein, 598 aa, Homo sapiens
MPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGGSEGRCAVCGDNASCQHYGVRTCEGCKGFFKRTVQKNAKYICLANKDCPVDKRRRNRCQFCRFQKCLAVGMVKEVVRTDSLKGRRGRLPSKPKQPPDASPANLLTSLVRAHLDSGPSTAKLDYSKFQELVLPHFGKEDAGDVQQFYDLLSGSLEVIRKWAEKIPGFAELSPADQDLLLESAFLELFILRLAYRSKPGEGKLIFCSGLVLHRLQCARGFGDWIDSILAFSRSLHSLLVDVPAFACLSALVLITDRHGLQEPRRVEELQNRIASCLKEHVAAVAGEPQPASCLSRLLGKLPELRTLCTQGLQRIFYLKLEDLVPPPPIIDKIFMDTLPF

SEQ ID NO: 31 - protein, 601 aa, Mus musculus
MPCIQAQYGTPATSPGPRDHLTGDPLALEFGKPTMDLASPETAPAAPATLPSFSTFMDGYTGEFDTFLYQLPGTTQPCSSACSSASSTSSSSSSATSPASASFKFEDFQVYGCYPGTLSGPLDETLSSSGSEYYGSPCSAPSPSTPNFQPSQLSPWDGSFGHFSPSQTYEGLWAWTEQLPKASSGPPPPPTFFSFSPPTGPSPSLAQSSLKLFPPPATHQLGEGESYSMPAAFPGLAPTSPNRDTSGILDAPVTSTKSRSGASGGSEGRCAVCGDNASCQHYGVRTCEGCKGFFKRTVQKSAKYICLANKDCPVDKRRRNRCQFCRFQKCLAVGMVKEVVRTDSLKGRRGRLPSKPKQPPDASPTNLLTSLIRAHLDSGPSTAKLDYSKFQELVLPRFGKEDAGDVQQFYDLLSGSLDVIRKWAEKIPGFIELCPGDQDLLLESAFLELFILRLAYRSKPGEGKLIFCSGLVLHQLQCARGFGDWIDNILAFSRSLHSLGVDVPAFACLSALVLITDRHGLQDPRRVEELQNRIASCLKEHMATVAGDPQPASCLSRLLGKLPELRTLCTQGLQRIFCLKLEDLVPPPPIVDKIFMDTLSF

SEQ ID NO: 32 - protein, 597 aa, RAT
MPCIQAQYGTPATSPGPRDHLTGDPLALEFSKPTMDLASPETAPTAPATLPSFSTFMDGGYTGEFDTFLYQLPGTAQPCSSASSTSSSSSSATSPASASFKFEDFQVYGCYPGTLSGPLDETLSSSGSDYYGSPCSAPSPPTPNFQPSQLSPWDGSFGHFSPSQTYEGLRVWTEQLPKASGPPPPPTFFSFSPPTGPSPSLAQSSLKLFPAPATHQLGEGESYSVPAAFPGLAPTSPNCDTSGILDAPVTSTKARSGSSGGSEGRCAVCGDNASCQHYGVRTCEGCKGFFKRTVQKSAKYICLANKDCPVDKRRRNRCQFCRFQKCLAVGMVKEVVRTDSLKGRRGRLPSKPKQPPDASPTNLLTSLIRAHLDSGPNTAKLDYSKFQELVLPRFGKEDAGDVQQFYDLLSGSLDVIRKWAEKIPGFIELSPGDQDLLLESAFLELFILRLAYRSKPGEGKLIFCSGLVLHRLQCARGFGDWIDNILAFSRSLHSLGVDVPAFACLSALVLITDRHGLQDPRRVEELQNRIASCLKEHMAAVAGDPQPASCLSRLLGKLPELRTLCTQGLQRIFCLKLEDLVPPPPIVDKIFMDTLSF

SEQ ID NO: 33 - protein, 535 aa, Homo sapiens [sequence omitted]

SEQ ID NO: 34 - protein, 597 aa, RAT
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPSTPGFQVQHSPMWDDPGSLHNFHQNYVATTHMIEQRKTPVSRLSLFSFKQSAPGTPVSSCQMRFDGPLHVPMNPEPAGSHHVVDGQTFAVPNPIRKPASMGFPGLQIGHASQLLDTQVPPSPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQDPSPPSPPGSDQCPRQTHVDSNPAMTSLDYSRFQANPDYQMSGDDTQHIQQFYDLLTGSMEIIRGWAEKIPGFADLPKASQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 35 - protein, 598 aa, Homo sapiens; identical to SEQ ID NO: 30

SEQ ID NO: 36 - DNA, 24 bp, Artificial Sequence
  Description of Artificial Sequence  Synthetic Primer
gtgatatttacctccaaatgccag